(19) United States
(12) Patent Application Publication
(10) Pub. No.: US 2013/0288310 A1
(43) Pub. Date: Oct. 31, 2013

(54) RECOMBINANT LIGNOCELLULOSE DEGRADATION ENZYMES FOR THE PRODUCTION OF SOLUBLE SUGARS FROM CELLULOSIC BIOMASS

(71) Applicants: Louis Clark, San Francisco, CA (US); Dipnath Baidyaroy, Fremont, CA (US)

(72) Inventors: Louis Clark, San Francisco, CA (US); Dipnath Baidyaroy, Fremont, CA (US)

(73) Assignee: Codexis, Inc, Redwood City, CA (US)

(21) Appl. No.: 13/818,393

(22) PCT Filed: Aug. 22, 2011
(86) PCT No.: PCT/US2011/048659

§ 371 Date: Jul. 15, 2013

Related U.S. Application Data
(60) Provisional application No. 61/376,188, filed on Aug. 23, 2010.

(51) Int. Cl. C12N 9/24

(52) U.S. Cl. CPC C12N 9/2402
USPC 435/99; 435/200; 435/197; 435/190; 435/189; 536/23.2; 435/320.1; 435/254.11; 435/254.2; 435/254.3; 435/254.7; 435/254.8; 435/254.4; 435/254.5; 435/254.9; 435/254.6; 435/254.22; 435/254.21; 435/254.23; 435/257.2; 435/252.3; 435/252.32; 435/252.33; 435/252.35; 435/252.31; 435/252.34

(57) ABSTRACT

The invention relates to C1 lignocellulose degradation enzyme nucleic acid and protein sequences and expression of recombinant C1 lignocellulose degradation enzymes. The invention provides methods for degrading a cellulosic biomass by contacting the biomass with a recombinant C1 lignocellulose degradation enzyme of the invention.

### CROSS-REFERENCE TO RELATED APPLICATIONS

The application claims benefit of U.S. provisional application No. 61/376,188, filed Aug. 23, 2010, which application is herein incorporated by reference for all purposes.

### FIELD OF THE INVENTION

The invention relates to expression of recombinant C1 enzymes involved in lignocellulose degradation and their use in the production of soluble sugars from cellulosic biomass.

### REFERENCE TO A “SEQUENCE LISTING,” A TABLE, OR A COMPUTER PROGRAM LISTING APPENDIX SUBMITTED AS A TEXT FILE

The ASCII text file SEQTXT_{—}90834-818631.TXT contains a sequence listing submitted under 37 CFR 1.821. The ASCII text file was created Aug. 22, 2011 and is 3,744,719 bytes in size. The material contained in this text file is herein incorporated by reference.

### BACKGROUND OF THE INVENTION

Cellulosic biomass is a significant renewable resource for the generation of sugars. Fermentation of these sugars can yield commercially valuable end-products, including biofuels and chemicals that are currently derived from petroleum. While the fermentation of simple sugars to ethanol is relatively straightforward, the efficient conversion of cellulosic biomass to fermentable sugars such as glucose is challenging. See, e.g., Ladisch et al., 1983, Enzyme Microb. Technol. 5:82. Cellulose may be pretreated chemically, mechanically or in other ways to increase the susceptibility of cellulose to hydrolysis. Such pretreatment may be followed by the enzymatic conversion of cellulose to glucose, cellobiose, cello-oligosaccharides and the like, using enzymes that specialize in breaking down the β-1-4 glycosidic bonds of cellulose. These enzymes are collectively referred to as “cellulases”.

Cellulases are divided into three sub-categories of enzymes: 1,4-β-D-glucan glucanohydrolase (“endoglucanase” or “EG”); 1,4-β-D-glucan cellobiohydrolase (“exoglucanase”, “cellobiohydrolase”, or “CBH”); and β-D-glucoside-glucohydrolase (“β-glucosidase”, “cellobiase” or “BG”). Endoglucanases randomly attack the interior parts and mainly the amorphous regions of cellulose. Exoglucanases incrementally shorten the glucan molecules by binding to the glucan ends and releasing mainly cellobiose units from the ends of the cellulose polymer. O-glucosidases split the cellobiose, a water-soluble β-1,4-linked dimer of glucose, into two units of glucose. Efficient production of cellulases for use in processing cellulosic biomass would reduce costs and increase the efficiency of production of biofuels and other commercially valuable compounds.

Other enzymes (“accessory enzymes” or “accessory proteins”) also participate in degradation of lignocellulose to obtain sugars. These enzymes include esterases, lipases, laccases, and other oxidative enzymes such as oxidoreductases, and the like.

In the context of this invention, the enzymes involved in degrading lignocellulose, e.g., a glycoside hydrolase or accessory enzyme, are collectively referred to as lignocellulose degradation enzymes.

### SUMMARY OF THE INVENTION

In one aspect, the invention provides a method of producing a lignocellulose degradation enzyme. The method involves culturing a cell comprising a recombinant polynucleotide sequence that encodes a C1 lignocellulose degradation enzyme comprising an amino acid sequence selected from SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, or SEQ ID NO: 178; or an amino acid sequence selected from SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, or SEQ ID NO: 720. In some embodiments, the recombinant polynucleotide sequence is operably linked to a promoter, or the polynucleotide sequence is present in multiple copies operably linked to a promoter, under conditions in which the lignocellulose degradation enzyme is produced. In some embodiments, the promoter is a heterologous promoter. In some embodiments, the lignocellulose degradation enzyme comprises a fragment that is less than the full-length of a polypeptide identified in Table 2. In some embodiments, the fragment comprises a number of contiguous amino acid residues of the sequence that is at least the number shown in Column 4 and less than the length shown in column 3 for that sequence. In some embodiments, the fragment comprises a number of contiguous amino acid residues of the sequence that is from 20 to 30 residues fewer in length than the number shown in Column 3. In some embodiments, the polypeptide comprises a lignocellulose degradation enzyme polypeptide that consists of an amino acid sequence set forth in Table 2. Optionally, the polynucleotide sequence encoding a C1 lignocellulose degradation enzyme of the invention has a nucleotide sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, or SEQ ID NO: 177; or a nucleotide sequence selected from SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, or SEQ ID NO: 719.

Also contemplated is a method of converting biomass substrates to a soluble sugar by combining a recombinant lignocellulose degradation enzyme made according to the invention with biomass substrates under conditions suitable for the production of the soluble sugar. In some embodiments the method includes the step of recovering the lignocellulose degradation enzyme from the medium in which the cell is cultured. In one aspect a composition comprising a recombinant lignocellulose degradation enzyme of the invention is provided.

In one aspect, the invention provides a method for producing soluble sugars from lignocellulose by contacting cellulosic biomass with a recombinant cell comprising a recombinant polynucleotide sequence that encodes a C1 lignocellulose degradation enzyme having an amino acid sequence selected from SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, or SEQ ID NO: 178; or an amino acid sequence selected from SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, or SEQ ID NO: 720; and where the polynucleotide sequence is operably linked to a promoter under conditions in which the enzyme is expressed and secreted by the cell and said cellulosic biomass is enzymatically converted using the lignocellulose degradation enzyme to a degradation product that produces soluble sugar. In some embodiments, the promoter is a heterologous promoter. In some embodiments, multiple copies of the polynucleotide sequence may be operably linked to a promoter. In some embodiments, the lignocellulose degradation enzyme comprises a fragment that is less than the full-length of a polypeptide identified in Table 2. In some embodiments, the fragment comprises a number of contiguous amino acid residues of the sequence that is at least the number shown in Column 4 and less than the length shown in column 3 for that sequence. In some embodiments, the fragment comprises a number of contiguous amino acid residues of the sequence that is from 20 to 30 residues less than the number shown in Column 3. In some embodiments, the polypeptide comprises a lignocellulose degradation enzyme polypeptide that consists of an amino acid sequence set forth in Table 2. Optionally, the polynucleotide encoding the lignocellulose degradation enzyme has a nucleic acid sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, or SEQ ID NO: 177; or a nucleic acid sequence selected from SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, or SEQ ID NO: 719.

In some embodiments of these methods the cell is a C1 cell and/or the heterologous promoter is a C1 promoter.

In one aspect, the invention provides a recombinant host cell comprising a recombinant polynucleotide sequence encoding a C1 lignocellulose degradation enzyme comprising an amino acid sequence selected from SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, or SEQ ID NO: 178; or an amino acid sequence selected from SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, or SEQ ID NO: 720; operably linked to a promoter, optionally a heterologous promoter. In some embodiments, the lignocellulose degradation enzyme comprises a fragment that is less than the full-length of a polypeptide identified in Table 2. In some embodiments, the fragment comprises a number of contiguous amino acid residues of the sequence that is at least the number shown in Column 4 and less than the length shown in column 3 for that sequence. In some embodiments, the fragment comprises a number of contiguous amino acid residues of the sequence that is from 20 to 30 residues fewer in length than the number shown in Column 3. In some embodiments, the polypeptide comprises a lignocellulose degradation enzyme polypeptide that consists of an amino acid sequence set forth in Table 2. Optionally, the recombinant polynucleotide has a nucleic acid sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, or SEQ ID NO: 177; or a nucleic acid sequence selected from SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, or SEQ ID NO: 719. In one embodiment the recombinant host cell expresses at least one other recombinant lignocellulose degradation enzyme, e.g., a cellulase enzyme or other enzyme involved in lignocellulose degradation. Also contemplated is a method of converting a biomass substrate to a soluble sugar, by combining the expression product from the recombinant cell with the biomass substrate under conditions suitable for the production of the soluble sugar.

In a further aspect, the invention provides a composition comprising a lignocellulose degradation enzyme having an amino acid sequence selected from the group of glycoside hydrolase amino acid sequences set forth in Table 1 or Table 2, and a cellulase, wherein the amino acid sequence of the cellulase is different from the glycoside hydrolase lignocellulose degradation enzyme of Table 1 or Table 2. In some embodiments, the glycoside hydrolase is set forth in Table 2. In some embodiments, the cellulase is derived from a filamentous fungal cell, e.g., a Trichoderma sp. or an Aspergillus sp.

### BRIEF DESCRIPTION OF THE TABLES

Tables 1 and 2 provide a description of the lignocellulose degradation enzymes of the invention. The SEQ ID NOs. shown in the Tables 1 and 2 refer to the nucleic acid and polypeptide sequences provided in the sequence appendix filed herewith, which is incorporated by reference. Table 1: Column 1, nucleic acid sequence identifier; Column 2, amino acid sequence identifier; Column 3, length of encoded polypeptide (number of amino acids); Column 4, indicates whether a secretion signal peptide is encoded by the gene; Column 5, Pfam domain structure present in the polypeptide; Column 6, enzyme class. Table 2: Column 1, nucleic acid sequence identifier; Column 2, amino acid sequence identifier; Column 3, length of encoded polypeptide (number of amino acids); Column 4, minimum fragment size (number of amino acids); Column 5, indicates whether a secretion signal peptide; Column 6, Pfam domain structure present in the polypeptide; Column 7, enzyme class. In the context of this invention, “a polynucleotide of” Table 1 or Table 2 refers to a polynucleotide that comprises a nucleotide sequence of a sequence identifier shown in Column 1; “a polypeptide of” or “lignocellulose degradation enzyme of” Table 1 or Table 2 refers to a polypeptide that comprises an amino acid sequence of a sequence identifier shown in Column 2.

### DETAILED DESCRIPTION OF THE INVENTION

### I. Definitions

The following definitions are provided to assist the reader. Unless otherwise defined, all terms of art are intended to have the meanings commonly understood by those of skill in the molecular biology and microbiology arts. In some cases, terms with commonly understood meanings are defined herein for clarity and/or for ready reference, and the inclusion of such definitions herein should not necessarily be construed to represent a substantial difference over the definition of the term as generally understood in the art.

As used in the context of this invention, the term “lignocellulose”, “cellulosic biomass”, and “biomass substrate” are used interchangeably. Lignocellulose is considered to be composed of cellulose (containing only glucose monomers); hemicellulose, which can contain sugar monomers other than glucose, including xylose, mannose, galactose, rhamnose, and arabinose; and lignin.

The term “lignocellulose degradation enzyme” is used herein to refer to enzymes that participate in lignocellulose degradation, and includes enzymes that degrade cellulose, lignin and hemicellulose. The term thus encompasses cellulases, xylanases, carbohydrate esterases, lipases, and enzymes that break down lignin including oxidases, peroxidases, laccases, etc. Glycoside hydrolases (GHs) are noted in Table 1 and Table 2 as a functional class. Other enzymes that are not glycoside hydrolases that participate in lignocellulose degradation are termed “accessory proteins” or “accessory enzymes” in Tables 1 and 2.

A “lignocellulose degradation product” as used herein can refer to an end product of lignocellulose degradation such as a soluble sugar, or to a product that undergoes further enzymatic conversion to an endproduct such as a soluble sugar. For example, a laccase can participate in the breakdown of lignin and although the laccase does not directly generate a soluble sugar, treatment of a lignocellulose biomass with laccase can result in an increase in the cellulose that is available for degradation. Similarly, various esterases can remove phenolic and acetyl groups from lignocellulose to aid in the production of soluble sugars. In typical lignocellulose degradation reactions, the cellulosic material is hydrolyzed to break down cellulose and/or hemicellulose to fermentable sugars, such as glucose, cellobiose, xylose, xylulose, arabinose, mannose, galactose, and/or soluble oligosaccharides.

“Glycoside hydrolases” (GHs), also referred to herein as “glycohydrolases”, (EC 3.2.1.) hydrolyze the glycosidic bond between two or more carbohydrates or between a carbohydrate and a non-carbohydrate moiety. The Carbohydrate-Active Enzymes database (CAZy) provides a continuously updated list of the glycoside hydrolase families. See, the web address “cazy.org/Glycoside-Hydrolases.html”.

The term “cellulase” refers to a category of enzymes capable of hydrolyzing cellulose (β-1,4-glucan or β-D-glucosidic linkages) to shorter oligosaccharides, cellobiose and/or glucose. Cellulases include 1,4-β-D-glucan glucanohydrolase (“endoglucanase” or “EG”); 1,4-β-D-glucan cellobiohydrolase (“exoglucanase”, “cellobiohydrolase”, or “CBH”); and β-D-glucoside-glucohydrolase (“β-glucosidase”, “cellobiase” or “BG”).

The term “β-glucosidase” or “cellobiase” used interchangeably herein means a β-D-glucoside glucohydrolase which catalyzes the hydrolysis of a sugar dimer, including but not limited to cellobiose, with the release of a corresponding sugar monomer. In one embodiment, a β-glucosidase is a β-glucoside glucohydrolase of the classification E.C. 3.2.1.21 which catalyzes the hydrolysis of cellobiose to glucose. Some of the β-glucosidases have the ability to also hydrolyze β-D-galactosides, β-L-arabinosides and/or β-D-fucosides and further some β-glucosidases can act on α-1,4-substrates such as starch. β-glucosidase activity may be measured by methods well known in the art, including the assays described hereinbelow. β-glucosidases include, but are not limited to, enzymes classified in the GH1, GH3, GH30, and GH116 GH families,

The term “β-glucosidase polypeptide” refers herein to a polypeptide having β-glucosidase activity.

The term “exoglucanase”, “exo-cellobiohydrolase” or “CBH” refers to a group of cellulase enzymes classified as E.C. 3.2.1.91. These enzymes hydrolyze cellobiose from the reducing or non-reducing end of cellulose. Exo-cellobiohydrolases include, but are not limited to, enzymes classified in the GH5, GH6, GH7, GH9, and GH48 GH families.

The term “endoglucanase” or “EG” refers to a group of cellulase enzymes classified as E.C. 3.2.1.4. These enzymes hydrolyze internal β-1,4 glucosidic bonds of cellulose. Endoglucanases include, but are not limited to, enzymes classified in the GH5, GH6, GH7, GH8, GH9, GH12, GH44, GH45, GH48, GH51, GH61, and GH74 GH families.

The term “xylanase” refers to a group of enzymes classified as E.C. 3.2.1.8 that catalyze the endo-hydrolysis of 1,4-beta-D-xylosidic linkages in xylans. Xylanases include, but are not limited to, enzymes classified in the GH5, GH8, GH10, GH11, and GH43 GH families.

The term “xylosidase” refers to a group of enzymes classified as E.C. 3.2.1.37 that catalyze the exo-hydrolysis of short beta (1 [symbol] 4)-xylooligosaccharides, to remove successive D-xylose residues from the non-reducing termini. Xylosidases include, but are not limited to, enzymes classified in the GH3, GH30, GH39, GH43, gH52, GH54, and GH116 GH families.

The term “arabinofuranosidase” refers to a group of enzymes classified as E.C. 3.2.1.55 that catalyze the hydrolysis of terminal non-reducing α-L-arabinofuranoside residues in α-L-arabinosides. The enzyme activity acts on α-L-arabinofuranosides, α-L-arabinans containing (1,3)- and/or (1,5)-linkages, arabinoxylans, and arabinogalactans. Arabinofuranosidases include, but are not limited to, enzymes classified in the GH3, GH43, GH51, GH54, and GH62 GH families.

The term “lignocellulose degradation enzyme activity” encompasses glycoside hydrolase enzyme activity, e.g., that hydrolyzes glycosidic bonds of cellulose, e.g., exoglucanase activity (CBH), endoglucanase (EG) activity and/or O-glucosidase activity, as well as the enzymatic activity of accessory enzymes such as carbohydrate esterases, e.g., aryl esterases, including feruloyl and coumaroyl esterases, acetyl esterases, lipases, phospholipases; laccases, oxidases, peroxidases, and the like.

The term “lignocellulose degradation enzyme polynucleotide” refers to a polynucleotide encoding a polypeptide having lignocellulose degradation enzyme activity.

As used herein, the term “isolated” refers to a nucleic acid, polynucleotide, polypeptide, protein, or other component that is partially or completely separated from components with which it is normally associated (other proteins, nucleic acids, cells, synthetic reagents, etc.).

The term “wildtype” as applied to a polypeptide (protein) means a polypeptide (protein) expressed by a naturally occurring microorganism such as bacteria or filamentous fungus. As applied to a microorganism, the term “wildtype” refers to the native, naturally occurring non-recombinant micro-organism.

A nucleic acid (such as a polynucleotide), and a polypeptide is “recombinant” when it is artificial or engineered. A cell is recombinant when it contains an artificial or engineered protein or nucleic acid or is derived from a recombinant parent cell. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

The term “culturing” or “cultivation” refers to growing a population of microbial cells under suitable conditions in a liquid or solid medium. In some embodiments, culturing refers to fermentative bioconversion of a cellulosic substrate to an end-product.

The term “contacting” refers to the placing of a respective enzyme in sufficiently close proximity to a respective substrate to enable the enzyme to convert the substrate to a product. Those skilled in the art will recognize that mixing solution of the enzyme with the respective substrate will effect contacting.

As used herein the term “transformed” or “transformation” used in reference to a cell means a cell has a non-native nucleic acid sequence integrated into its genome or as an episomal plasmid that is maintained through multiple generations.

The term “introduced” in the context of inserting a nucleic acid sequence into a cell means transfected, transduced or transformed (collectively “transformed”) and prokaryotic cell wherein the nucleic acid is incorporated into the genome of the cell.

As used herein, “C1” refers to a fungal strain described by Garg, A., 1966, “An addition to the genus Chrysosporium corda” Mycopathologia 30: 3-4. “Chrysosporium lucknowense” includes the strains described in U.S. Pat. Nos. 6,015,707, 5,811,381 and 6,573,086; US Pat. Pub. Nos. 2007/0238155, US 2008/0194005, US 2009/0099079; International Pat. Pub. Nos., WO 2008/073914 and WO 98/15633, and include, without limitation, Chrysosporium lucknowense Garg 27K, VKM-F 3500 D (Accession No. VKM F-3500-D), C1 strain UV13-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Although initially described as Chrysosporium lucknowense, C1 may currently be considered a strain of Myceliophthora thermophilia. Exemplary C1 strains include modified organisms in which one or more endogenous genes or sequences has been deleted or modified and/or one or more heterologous genes or sequences has been introduced, such as UV18#100.f (CBS Accession No. 122188). Derivatives include UV18#100.f Δalp1, UV18#100.f Δpyr5 Δalp1, UV18#100.f Δalp1 Δpep4 Δalp2, UV18#100.f Δpyr5 Δalp1 Δpep4 Δalp2 and UV18#100.f Δpyr4 Δpyr5 Δalp1 Δpep4 Δalp2, as described in WO2008073914, incorporated herein by reference.

The term “operably linked” refers herein to a configuration in which a control sequence is appropriately placed at a position relative to the coding sequence of the DNA sequence such that the control sequence influences the expression of RNA encoding a polypeptide.

When used herein, the term “coding sequence” is intended to cover a nucleotide sequence that directly specifies the amino acid sequence of its protein product. The boundaries of the coding sequence are generally determined by an open reading frame, which usually begins with the ATG start codon.

A promoter or other nucleic acid control sequence is “heterologous”, when it is operably linked to a sequence encoding a protein sequence with which the promoter is not associated in nature. For example, in a recombinant construct in which the C1 Cbh1a promoter is operably linked to a protein coding sequence other than the C1 Cbh1a gene the promoter is heterologous. For example, in a construct comprising a C1 Cbh1a promoter operably linked to a C1 nucleic acid encoding a lignocellulose degradation enzyme of Table 1 or Table 2, the promoter is heterologous. Similarly, a polypeptide sequence such as a secretion signal sequence, is “heterologous” to a polypeptide sequence when it is linked to a polypeptide sequence that it is not associated with in nature.

As used herein, the term “expression” includes any step involved in the production of the polypeptide including, but not limited to, transcription, post-transcriptional modification, translation, post-translational modification, and secretion.

The term “expression vector” refers herein to a DNA molecule, linear or circular, that comprises a segment encoding a polypeptide of the invention, and which is operably linked to additional segments that provide for its transcription.

A polypeptide is “enzymatically active” when it has a lignocellulose degradation enzyme activity. Thus, a polypeptide of the invention may have a glycoside hydrolase activity, or another enzymatic activity shown in Table 1 or Table 2.

The term “pre-protein” refers to a secreted protein with an amino-terminal signal peptide region attached. The signal peptide is cleaved from the pre-protein by a signal peptidase prior to secretion to result in the “mature” or “secreted” protein.

As used herein, a “start codon” is the ATG codon that encodes the first amino acid residue (methionine) of a protein.

### II. Introduction

The fungus C1 produces a variety of enzymes that act in concert to catalyze decrystallization and hydrolysis of cellulose to yield soluble sugars. The present invention is based on the discovery and characterization of C1 genes encoding lignocellulose degradation enzymes that can be used to facilitate lignocellulose degradation.

The C1 lignocellulose degradation enzymes of the invention, and polynucleotides encoding them, may be used in a variety of applications in which lignocellulose degradation enzyme activity is desired, such as those described hereinbelow. For simplicity, and as will be apparent from context, references to a “C1 lignocellulose degradation enzyme” and the like may be used to refer both to a secreted mature form of the enzyme protein and to the pre-protein form.

In various embodiments of the invention, a recombinant nucleic acid sequence is operably linked to a promoter. In one embodiment, a nucleic acid sequence encoding a C1 lignocellulose degradation enzyme comprising an amino acid sequence selected from SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, or SEQ ID NO: 178; or an amino acid sequence selected from SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, or SEQ ID NO: 720 is operably linked to a promoter not associated with the enzyme in nature (i.e., a heterologous promoter), to, for example, improve expression efficiency of the cellulose degradation enzyme protein when expressed in a host cell. In one embodiment the host cell is a fungus, such as a filamentous fungus. In one embodiment the host cell is a C1 cell. In one embodiment the host cell is a C1 cell and the promoter is a heterologous C1 promoter.

A C1 lignocellulose degradation enzyme expression system comprising one or more lignocellulose degradation enzymes of Table 1 or Table 2 is particularly useful for production of soluble carbohydrates from cellulosic biomass. In one aspect the invention relates to a method of producing a soluble sugar, e.g., glucose, xylose, etc., by contacting a composition comprising cellulosic biomass with a recombinantly expressed C1 enzyme of Table 1 or Table 2, e.g., a glycohydrolase of Table 1 or Table 2, under conditions in which the biomass is enzymatically degraded. In some embodiments, the cellulosic biomass is contacted with one or more accessory enzymes of Table 1 or Table 2. Purified or partially purified recombinant lignocellulose degradation enzyme may be contacted with the cellulosic biomass. In one aspect of the present invention, said “contacting” comprises culturing a recombinant host cell in a medium that contains biomass produced from a lignocellulosic feedstock, where the recombinant cell comprises a sequence encoding a C1 lignocellulose degradation enzyme of Table 1 or Table 2 operably linked to a heterologous promoter or to a homologous promoter when said sequence is present in multiple copies per cell.

In some embodiments, a lignocellulose degradation enzyme of the invention comprises a fragment of a polypeptide having an amino acid sequence set forth in Table 2 (i.e., an amino acid sequence set forth in SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, or SEQ ID NO: 720), where the fragment comprises a number of contiguous amino acid residues of the sequence that is at least the number shown in Column 4 and less than the length shown in column 3 for that sequence. In some embodiments, the fragment comprises a number of contiguous amino acid residues of the sequence that is from 20 to 30 residues less fewer in length than the number shown in Column 3.

In another aspect of the invention, a heterologous C1 signal peptide may be fused to the amino terminus of a lignocellulose degradation enzyme polypeptide of Table 1 or Table 2 to improve secretion, stability, or other properties of the polypeptide when expressed in a host cell, e.g., a fungal cell such as C1.

In some embodiments, a lignocellulose degradation enzyme of the invention is a glycohydrolase that has an amino acid sequence identified in Table 2 and comprises a GH3, GH5, GH6, GH7, GH10, GH11, GH62, GH30, or GH43 family Pfam domain.

In some embodiments, a lignocellulose degradation enzyme of the invention is a cellobiohydrolase or endoglucanase that is a member of a GH5, GH6, or GH7 family and has an amino acid sequence of a glycohydrolase set forth in Table 2. In some embodiments, a lignocellulose degradation enzyme of the invention is a β-glucosidase that is a member of a GH3 or GH30 family and has an amino acid sequence of a glycohydrolase set forth in Table 2. In some embodiments, a lignocellulose degradation enzyme of the invention is a β-xylosidase that is a member of a GH3, GH30, or GH43 family and has an amino acid sequence of a glycohydrolase set forth in Table 2. In some embodiments, a lignocellulose degradation enzyme of the invention is a xylanase that is a member of a GH5, GH10, GH11, or GH43 family and has an amino acid sequence of a glyocohydrolase set forth in Table 2. In some embodiments, a lignocellulose degradation enzyme of the invention is an arabinofuranosidase that is a member of a GH3, GH43, or GH62 family and has an amino acid sequence of a glyocohydrolase set forth in Table 2.

Various aspects of the invention are described in the following sections.

### III. Properties of Lignocellulose Degradation Enzyme Proteins for Use in Methods of the Invention

In one aspect, the invention provides a method for expressing a lignocellulose degradation enzyme by culturing a host cell comprising a vector comprising a nucleic acid sequence encoding a C1 polypeptide sequence of Table 1 or Table 2 operably linked to a heterologous promoter, under conditions in which the lignocellulose degradation protein or an enzymatically active fragment thereof is expressed. Generally, the expressed protein comprises a signal peptide which is removed in the secretion process. In some embodiments, the nucleic acid sequence is a nucleic acid sequence of Table 1 or Table 2.

In some embodiments the lignocellulose degradation enzyme polypeptide of Table 1 or Table 2 includes additional sequences that do not alter the activity of the encoded enzyme. For example, the lignocellulose degradation enzyme polypeptide may be linked to an epitope tag or to other sequence useful in purification.

### Signal Peptide

In general, lignocellulose degradation enzyme polypeptides are secreted from the host cell in which they are expressed (e.g., C1) and are expressed as a pre-protein including a signal peptide, i.e., an amino acid sequence linked to the amino terminus of a polypeptide that directs the encoded polypeptide into the cell secretory pathway. In one embodiment, the signal peptide is an endogenous C1 signal peptide of a polypeptide sequence of Table 1 or Table 2. In other embodiments, signal peptide from other C1 secreted proteins are used.

Other signal peptides may be used, depending on the host cell and other factors. Effective signal peptide coding regions for filamentous fungal host cells include but are not limited to the signal peptide coding regions obtained from Aspergillus oryzae TAKA amylase, Aspergillus niger neutral amylase, Aspergillus niger glucoamylase, Rhizomucor miehei asparatic proteinase, Humicola insolens cellulase, Humicola lanuginosa lipase, and T. reesei cellobiohydrolase II. For example, a C1 lignocellulose degradation enzyme sequence may be used with a variety of filamentous fungal signal peptides known in the art. Useful signal peptides for yeast host cells also include those from the genes for Saccharomyces cerevisiae alpha-factor and Saccharomyces cerevisiae invertase. Still other useful signal peptide coding regions are described by Romanos et al., 1992, Yeast 8:423-488. Effective signal peptide coding regions for bacterial host cells are the signal peptide coding regions obtained from the genes for Bacillus NClB 11837 maltogenic amylase, Bacillus stearothermophilus alpha-amylase, Bacillus licheniformis subtilisin, Bacillus licheniformis β-lactamase, Bacillus stearothermophilus neutral proteases (nprT, nprS, nprM), and Bacillus subtilis prsA. Further signal peptides are described by Simonen and Palva, 1993, Microbiol Rev 57: 109-137. Variants of these signal peptides and other signal peptides are also suitable.

### Enzyme Activity

The activity of lignocellulose degradation enzymes of the invention, e.g., to evaluate an expression system, assess activity levels in an enzyme mixture comprising the enzyme, etc. can be determined by methods well known in the art for each of the various glycoside hydrolases or accessory proteins of Table 1 or Table 2. For example, esterase activity can be determined by measuring the ability of an enzyme to hydrolyze an ester. Glycoside hydrolase activity can be determined using known assays to measure the hydrolysis of glyosidic linkages. Enzymatic activity of oxidases and oxidoreductases can be assessed using techniques to measure oxidation of known substrates.

Thus, for example, α-arabinofuranosidase enzymatic activity can be measured by measuring the release of p-nitrophenol by the action of α-arabinofuranosidase on p-nitrophenyl α-L-arabinofuranoside. Xylosidase activity can be assessed, e.g., by measuring the release of xylose by the action of a xylosidase on xylobiose. Xylanase activity can be assessed using known assays. For example, xylanolytic activity can be assayed based on production of reducing sugars from polymeric 4-O-methyl glucuronoxylan as described in Bailey, et al., 1992, Journal of Biotechnol. 23(3): 257-270. β-glucosidase activity can be determined, e.g., by using a colorimetric pNPG (p-nitrophenyl-β-D-glucopyranoside)-based assay that measure the enzyme-mediated conversion of pNPG to p-nitrophenol or by using an assay in which cellobiose is the substrate. Endoglucanase activity may be determined, e.g., either by a colorimetric para-nitrophenyl-β-D-cellobioside (pNPC) assay, or a cellulose assay. Cellobiohydrolase activity can be determined, e.g., by assessing release of water-soluble reducing sugar from cellulose as measured by the PAHBAH method of Lever et al., 1972, Anal. Biochem. 47: 273-279.)

### IV. Lignocellulose Degradation Enzyme Polynucleotides and Expression Systems

The present invention provides polynucleotide sequences that encode C1 lignocellulose degradation enzymes. The C1 cDNA sequences encoding lignocellulose degradation enzymes are each identified by a sequence identifier in Tables 1 and 2 with reference to the appended sequence listing. These sequences encode the respective polypeptides in Table 1 and Table 2, which are each identified by a sequence identifier with reference to the appended sequence listing. Those having ordinary skill in the art will readily appreciate that due to the degeneracy of the genetic code, a multitude of nucleotide sequences encoding cellulose degradation enzyme polypeptides of Table 1 or Table 2 exist. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence. The invention contemplates and provides each and every possible variation of nucleic acid sequence encoding a lignocellulose degradation polypeptide of the invention that could be made by selecting combinations based on possible codon choices.

A DNA sequence may also be designed for high codon usage bias codons (codons that are used at higher frequency in the protein coding regions than other codons that code for the same amino acid). The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. In particular, a DNA sequence can be optimized for expression in a particular host organism. See GCG CodonPreference, Genetics Computer Group Wisconsin Package; Codon W, John Peden, University of Nottingham; McInerney, J. O. 1998, Bioinformatics 14:372-73; Stenico et al., 1994, Nucleic Acids Res. 222437-46; Wright, F., 1990, Gene 87:23-29; Wada et al., 1992, Nucleic Acids Res. 20:2111-2118; Nakamura et al., 2000, Nucl. Acids Res. 28:292, all of which are incorporated herein be reference.

### Expression Vectors

The present invention makes use of recombinant constructs comprising a sequence encoding a lignocellulose degradation enzyme of Table 1 or Table 2. In a particular aspect, the present invention provides an expression vector encoding a glycohydrolase of Table 1 or Table 2 wherein the polynucleotide encoding the glycohydrolase is operably linked to a heterologous promoter. In another aspect, the invention provides an expression vector encoding an accessory enzyme of Table 1 or Table 2. Expression vectors of the present invention may be used to transform an appropriate host cell to permit the host to express the lignocellulose degradation protein. Methods for recombinant expression of proteins in fungi and other organisms are well known in the art, and any number of expression vectors are available or can be constructed using routine methods. See, e.g., Tkacz and Lange, 2004, ADVANCES IN FUNGAL BIOTECHNOLOGY FOR INDUSTRY, AGRICULTURE, AND MEDICINE, KLUWER ACADEMIC/PLENUM PUBLISHERS. New York; Zhu et al., 2009, Construction of two Gateway vectors for gene expression in fungi Plasmid 6:128-33; Kavanagh, K. 2005, FUNGI: BIOLOGY AND APPLICATIONS Wiley, all of which are incorporated herein by reference.

Nucleic acid constructs of the present invention comprise a vector, such as, a plasmid, a cosmid, a phage, a virus, a bacterial artificial chromosome (BAC), a yeast artificial chromosome (YAC), and the like, into which a nucleic acid sequence encoding a lignocellulose degradation enzyme protein of Table 1 or Table 2 has been inserted. The nucleic acids can be incorporated into any one of a variety of expression vectors suitable for expressing a polypeptide. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, which is replicable and viable in the relevant host can be used.

In an aspect of this embodiment, the construct further comprises regulatory sequences, including, for example, a promoter, operably linked to the protein encoding sequence. Large numbers of suitable vectors and promoters are known to those of skill in the art. The construct may optionally include nucleotide sequences to facilitate integration into a host genome and/or results in amplification of construct copy number in vivo.

### Promoter/Gene Constructs

As discussed above, to obtain high levels of expression in a particular host it is often useful to express C1 lignocellulose degradation enzymes under control of a heterologous promoter. Typically a promoter sequence may be operably linked to the 5′ region of the C1 lignocellulose degradation protein coding sequence. It will be recognized that in making such a construct it is not necessary to define the bounds of a minimal promoter. Instead, the DNA sequence 5′ to the C1 lignocellulose degradation gene start codon can be replaced with DNA sequence that is 5′ to the start codon of a given heterologous gene (e.g., a C1 sequence from another gene, or a promoter from another organism). This 5′ “heterologous” sequence thus includes, in addition to the promoter elements per se, a transcription start signal and the sequence of the 5′ untranslated portion of the transcribed chimeric mRNA. Thus, the promoter-gene construct and resulting mRNA will comprise a sequence encoding a lignocellulose degradation enzyme of Table 1 or Table 2 and a heterologous 5′ sequence upstream to the start codon of the sequence encoding the lignocellulose degradation enzyme. In some, but not all, cases the heterologous 5′ sequence will immediately abut the start codon of the polynucleotide sequence encoding the cellulose degradation protein. In some embodiments, gene constructs may be employed in which a polynucleotide encoding a lignocellulose degradation enzyme of Table 1 or Table 2 is present in multiple copies. Such embodiments, may employ the endogenous promoter for the lignocellulose degradation gene or may employ a heterologous promoter.

In one embodiment, the C1 lignocellulose degradation enzyme is expressed as a pre-protein including the naturally occurring signal peptide of a lignocellulose degradation enzyme in Table 1 or Table 2.

In one embodiment of the gene construct of the present invention, the C1 lignocellulose degradation enzyme is expressed from the construct as a pre-protein with a heterologous signal peptide.

In some embodiments the heterologous promoter is operably linked to a lignocellulose degradation enzyme cDNA nucleic acid sequence of Table 1 or Table 2.

Examples of useful promoters for expression of lignocellulose degradation enzymes include promoters from fungi. For example, promoter sequences that drive expression of homologous or orthologous genes from other organisms may be used. For example, a fungal promoter from a gene encoding cellobiohydrolase may be used.

Examples of other suitable promoters useful for directing the transcription of the nucleotide constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nunberg et al., Mol. Cell Biol., 4:2306-2315 (1984), Boel et al., EMBO J. 3:1581-1585 ((1984) and EPA 137280, all of which are incorporated herein by reference), and mutant, truncated, and hybrid promoters thereof. In a yeast host, useful promoters can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are described by Romanos et al., 1992, Yeast 8:423-488. Promoters associated with chitinase production in fungi may be used. See, e.g., Blaiseau and Lafay, 1992, Gene 120243-248 (filamentous fungus Aphanocladium album); Limon et al., 1995, Curr. Genet, 28:478-83 (Trichoderma harzianum), both of which are incorporated herein by reference.

Promoters known to control expression of genes in prokaryotic or eukaryotic cells or their viruses that can be used in some embodiments of the invention include SV40 promoter, E. coli lac or trp promoter, phage lambda P_{L} promoter, tac promoter, T7 promoter, and the like. In bacterial host cells, suitable promoters include the promoters obtained from the E. coli lac operon, Streptomyces coelicolor agarase gene (dagA), Bacillus subtilis levansucranse gene (sacB), Bacillus licheniformis alpha-amylase gene (amyl), Bacillus stearothermophilus maltogenic amylase gene (amyM), Bacillus amyloliquefaciens alpha-amylase gene (amyQ), Bacillus subtilis xylA and xylB genes and prokaryotic β-lactamase gene.

An expression vector can contain other sequences, for example, an expression vector may optionally contain a ribosome binding site for translation initiation, and a transcription terminator. The vector also optionally includes appropriate sequences for amplifying expression, e.g., an enhancer.

In addition, expression vectors that encodes a cellulose degradation enzyme of the invention optionally contain one or more selectable marker genes to provide a phenotypic trait for selection of transformed host cells. Suitable marker genes include those coding for antibiotic resistance such as, ampicillin (ampR), kanamycin, chloramphenicol, or tetracycline resistance. Further examples include the antibiotics spectinomycin (e.g., the aada gene); streptomycin, e.g., the streptomycin phosphotransferase (SPT) gene coding for streptomycin resistance; the neomycin phosphotransferase (NPTII) gene encoding kanamycin or geneticin resistance; the hygromycin phosphotransferase (HPT) gene coding for hygromycin resistance. Additional selectable marker genes include dihydrofolate reductase or neomycin resistance for eukaryotic cell culture, and tetracycline or ampicillin resistance in E. coli. Selecteable markers for fungi include markers for resistance to HPT, phleomycin, benomyl, and acetamide.

### Synthesis and Manipulation of LignoCellulose Degradation Enzyme Polynucleotides

Polynucleotides encoding a lignocellulose degradation enzyme of Table 1 or Table 2 can be prepared using methods that are well known in the art. For example, individual oligonucleotides may be individually synthesized, then joined (e.g., by enzymatic or chemical ligation methods, or polymerase-mediated methods) to form essentially any desired continuous sequence. Chemical synthesis of oligonucleotides can be performed using, for example, the classical phosphoramidite method described by Beaucage, et al., 1981, Tetrahedron Letters, 22:1859-69, or the method described by Matthes, et al., 1984, EMBO J. 3:801-05, both of which are incorporated herein by reference. These methods are typically practiced in automated synthetic methods. In a chemical synthesis method, oligonucleotides are synthesized, e.g., in an automatic DNA synthesizer, purified, annealed, ligated and cloned in appropriate vectors. Further, essentially any nucleic acid can be custom ordered from any of a variety of commercial sources.

General texts that describe molecular biological techniques that are useful herein, including the use of vectors, promoters, protocols sufficient to direct persons of skill through in vitro amplification methods, including the polymerase chain reaction (PCR) and the ligase chain reaction (LCR), and many other relevant methods, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”), all of which are incorporated herein by reference. Reference is made to Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564, all of which are incorporated herein by reference. Methods for cloning in vitro amplified nucleic acids are described in Wallace et al., U.S. Pat. No. 5,426,039, which is incorporated herein by reference.

### Expression Hosts

The present invention also provides engineered (recombinant) host cells that are transformed with an expression vector or DNA construct encoding a lignocellulose degradation enzyme of Table 1 or Table 2. As used herein, a genetically modified or recombinant host cell includes the progeny of said host cell that comprises a lignocellulose degradation enzyme polynucleotide that encodes a recombinant polypeptide of Table 1 or Table 2. In some embodiments, the genetically modified or recombinant host cell is a eukaryotic cell. Suitable eukaryotic host cells include, but are not limited to, fungal cells, algal cells, insect cells, and plant cells. In some cases host cells may be modified to increase protein expression, secretion or stability, or to confer other desired characteristics. Cells (e.g., fungi) that have been mutated or selected to have low protease activity are particularly useful for expression. For example, C1 strains in which the alp1 (alkaline protease) locus has been deleted or disrupted may be used. Many expression hosts can be employed in the invention, including fungal host cell, such as yeast cells and filamentous fungal cells; algal host cells; and prokaryotic cells, including gram positive, gram negative and gram-variable bacterial cells. Examples are listed below.

Suitable fungal host cells include, but are not limited to, Ascomycota, Basidiomycota, Deuteromycota, Zygomycota, Fungi imperfecti. Particularly preferred fungal host cells are yeast cells and filamentous fungal cells. The filamentous fungal host cells of the present invention include all filamentous forms of the subdivision Eumycotina and Oomycota. (see, for example, Hawksworth et al., In Ainsworth and Bisby's Dictionary of The Fungi, 8^{th} edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungal host cells of the present invention are morphologically distinct from yeast.

In some embodiments the filamentous fungal host cell may be a cell of a species of, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothia, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, or teleomorphs, or anamorphs, and synonyms or taxonomic equivalents thereof.

In some embodiments of the invention, the filamentous fungal host cell is of the Aspergillus species, Ceriporiopsis species, Chrysosporium species, Corynascus species, Fusarium species, Humicola species, Neurospora species, Penicillium species, Tolypocladium species, Tramates species, or Trichoderma species.

In some embodiments of the invention, the filamentous fungal host cell is of the Trichoderma species, e.g., T. longibrachiatum, T. viride (e.g., ATCC 32098 and 32086), Hypocrea jecorina or T. reesei (NRRL 15709, ATTC 13631, 56764, 56765, 56466, 56767 and RL-P37 and derivatives thereof—See Sheir-Neiss et al., 1984, Appl. Microbiol. Biotechnology, 20:46-53, which is incorporated herein by reference), T. koningii, and T. harzianum. In addition, the term “Trichoderma” refers to any fungal strain that was previously classified as Trichoderma or currently classified as Trichoderma.

In some embodiments of the invention, the filamentous fungal host cell is of the Aspergillus species, e.g., A. awamori, A. fumigatus, A. japonicus, A. nidulans, A. niger, A. aculeatus, A. foetidus, A. oryzae, A. sojae, and A. kawachi. (Reference is made to Kelly and Hynes, 1985, EMBO J. 4, 475479; NRRL 3112, ATCC 11490, 22342, 44733, and 14331; Yelton et al., 1984, Proc. Natl. Acad. Sci. USA, 81, 1470-1474; Tilburn et al., 1982, Gene 26, 205-221; and Johnston et al., 1985, EMBO J. 4, 1307-1311, all of which are incorporated herein by reference).

In some embodiments of the invention, the filamentous fungal host cell is of the Fusarium species, e.g., F. bactridioides, F. cerealis, F. crookwellense, F. culmorum, F. graminearum, F. graminum. F. oxysporum, F. roseum, and F. venenatum. In some embodiments of the invention, the filamentous fungal host cell is of the Neurospora species, e.g., N. crassa. Reference is made to Case, M. E. et al., (1979) Proc. Natl. Acad. Sci. USA, 76, 5259-5263; U.S. Pat. No. 4,486,553; and Kinsey, J. A. and J. A. Rambosek (1984) Molecular and Cellular Biology 4, 117-122, all of which are incorporated herein by reference. In some embodiments of the invention, the filamentous fungal host cell is of the Humicola species, e.g., H. insolens, H. grisea, and H. lanuginosa. In some embodiments of the invention, the filamentous fungal host cell is of the Mucor species, e.g., M. miehei and M. circinelloides. In some embodiments of the invention, the filamentous fungal host cell is of the Rhizopus species, e.g., R. oryzae and R. niveus. In some embodiments of the invention, the filamentous fungal host cell is of the Penicillum species, e.g., P. purpurogenum, P. chrysogenum, and P. verruculosum. In some embodiments of the invention, the filamentous fungal host cell is of the Thielavia species, e.g., T. terrestris. In some embodiments of the invention, the filamentous fungal host cell is of the Tolypocladium species, e.g., T. inflatum and T. geodes. In some embodiments of the invention, the filamentous fungal host cell is of the Trametes species, e.g., T. villosa and T. versicolor.

In some embodiments of the invention, the filamentous fungal host cell is of the Chrysosporium species, e.g., C1, C. lucknowense, C. keratinophilum, C. tropicum, C. merdarium, C. inops, C. pannicola, and C. zonatum. In a particular embodiment the host is C1.

In the present invention a yeast host cell may be a cell of a species of, but not limited to Candida, Hansenula, Saccharomyces, Schizosaccharomyces, Pichia, Kluyveromyces, and Yarrowia. In some embodiments of the invention, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, and Yarrowia lipolytica.

In some embodiments on the invention, the host cell is an algal such as, Chlamydomonas (e.g., C. reinhardtii) and Phormidium (P. sp. ATCC29409).

In other embodiments, the host cell is a prokaryotic cell. Suitable prokaryotic cells include gram positive, gram negative and gram-variable bacterial cells. The host cell may be a species of, but not limited to, Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Mycobacterium, Neisseria, Pantoea, Pseudornonas, Prochlorococcus, Rhodobacter, Rhodopseudomonas, Rhodopseudomonas, Roseburia, Rhodospirillum, Rhodococcus, Scenedesmus, Streptomyces, Streptococcus, Synechococcus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia and Zymomonas.

In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, and Zymomonas.

In yet other embodiments, the bacterial host strain is non-pathogenic to humans. In some embodiments the bacterial host strain is an industrial strain. Numerous bacterial industrial strains are known and suitable in the present invention.

In some embodiments of the invention the bacterial host cell is of the Agrobacterium species, e.g., A. radiobacter, A. rhizogenes, and A. rubi. In some embodiments of the invention the bacterial host cell is of the Arthrobacter species, e.g., A. aurescens, A. citreus, A. globformis, A. hydrocarboglutamicus, A. mysorens, A. nicotianae, A. paraffineus, A. protophonniae, A. roseoparqffinus, A. sulfureus, and A. ureafaciens. In some embodiments of the invention the bacterial host cell is of the Bacillus species, e.g., B. thuringiensis, B. anthracis, B. megaterium, B. subtilis, B. lentus, B. circulans, B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. Some preferred embodiments of a Bacillus host cell include B. subtilis, B. licheniformis, B. megaterium, B. stearothermophilus and B. amyloliquefaciens. In some embodiments the bacterial host cell is of the Clostridium species, e.g., C. acetobutylicum, C. tetani E88, C. lituseburense, C. saccharobutylicum, C. perfringens, and C. beijerinckii. In some embodiments the bacterial host cell is of the Corynebacterium species e.g., C. glutamicum and C. acetoacidophilum. In some embodiments the bacterial host cell is of the Escherichia species, e.g., E. coli. In some embodiments the bacterial host cell is of the Erwinia species, e.g., E. uredovora, E. carotovora, E. ananas, E. herbicola, E. punctata, and E. terreus. In some embodiments the bacterial host cell is of the Pantoea species, e.g., P. citrea, and P. agglomerans. In some embodiments the bacterial host cell is of the Pseudomonas species, e.g., P. putida, P. aeruginosa, P. mevalonii, and P. sp. D-01 10. In some embodiments the bacterial host cell is of the Streptococcus species, e.g., S. equisimiles, S. pyogenes, and S. uberis. In some embodiments the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans. In some embodiments the bacterial host cell is of the Zymomonas species, e.g., Z. mobilis, and Z. lipolytica.

Strains that may be used in the practice of the invention including both prokaryotic and eukaryotic strains, are readily accessible to the public from a number of culture collections such as American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Host cells may be genetically modified to have characteristics that improve protein secretion, protein stability or other properties desirable for expression and/or secretion of a protein. Genetic modification can be achieved by genetic engineeriing techniques or using classical microbiological techniques, such as chemical or UV mutagenesis and subsequent selection. A combination of recombinant modification and classical selection techniques may be used to produce the organism of interest. Using recombinant technology, nucleic acid molecules can be introduced, deleted, inhibited or modified, in a manner that results in increased yields of a lignocellulose degradation enzyme of the invention, e.g., a glycohydrolase of the invention, within the organism or in the culture. For example, knock out of pyr5 function results in a cell with a pyrimidine deficient phenotype.

### Transformation

Introduction of a vector or DNA construct into a host cell can be effected by calcium phosphate transfection, DEAE-Dextran mediated transfection, electroporation, or other common techniques (See Davis et al., 1986, Basic Methods in Molecular Biology, which is incorporated herein by reference). Transformation of C1 host cells is known in the art (see, e.g., US 2008/0194005 which is incorporated herein by reference).

### Culture Conditions

The engineered host cells can be cultured in conventional nutrient media modified as appropriate for activating promoters, selecting transformants, or amplifying the lignocellulose degradation enzyme polynucleotide. Culture conditions, such as temperature, pH and the like, are those previously used with the host cell selected for expression, and will be apparent to those skilled in the art. As noted, many references are available for the culture and production of many cells, including cells of bacterial, plant, animal (especially mammalian) and archaebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

Culture conditions for C1 host cells are known in the art and can be readily determined by one of skill. See, e.g., US 2008/0194005, US 20030187243, WO 2008/073914 and WO 01/79507, which are incorporated herein by reference.

### V. Production and Recovery of Lignocellulose Degradation Enzyme Polypeptides

The present invention is directed to a method of making a lignocellulose degradation enzyme having an amino acid sequence of Table 1 or Table 2, the method comprising providing a host cell transformed with a polynucleotide encoding the enzyme, e.g., a nucleic acid of Table 1 or Table 2; culturing the transformed host cell in a culture medium under conditions in which the host cell expresses the encoded enzyme; and optionally recovering or isolating the expressed lignocellulose degradation ezyme, or recovering or isolating the culture medium containing the expressed enzyme. The method further provides optionally lysing the transformed host cells after expressing the lignocellulose degradation enzyme and optionally recovering or isolating the expressed enzyme from the cell lysate.

In a further embodiment, the present invention provides a method of over-expressing (i.e., making,) a lignocellulose degradation enzyme having an amino acid sequence of Table 1 or Table 2 comprising: (a) providing a recombinant C1 host cell comprising a nucleic acid construct, wherein the nucleic acid construct comprises a polynucleotide sequence that encodes a C1 lignocellulose degradation enzyme of Table 1 or Table 2 and the nucleic acid construct optionally also comprises a polynucleotide sequence encoding a signal peptide at the amino terminus of the lignocellulose degradation enzyme, wherein the polynucleotide sequence encoding the enzyme and optional signal peptide is operably linked to a heterologous promoter; and (b) culturing the host cell in a culture medium under conditions in which the host cell expresses the encoded lignocellulose degradation enzyme, wherein the level of expression of protein from the host cell is greater, preferably at least about 2-fold greater, than that from wildtype C1 cultured under the same conditions. The signal peptide employed in this method may be any heterologous signal peptide known in the art or may be a wildtype signal peptide of a sequence set forth in Table 1 or Table 2. In some embodiments, the level of overexpression is at least about 5-fold, 10-fold, 12-fold, 15-fold, 20-fold, 25-fold, 30-fold, or 35-fold greater than expression of the enzyme from wildtype C1.

Typically, recovery or isolation of the lignocellulose degradation polypeptide is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including freeze-thaw cycling, sonication, mechanical disruption, or use of cell lysing agents, or other methods, which are well known to those skilled in the art.

The resulting polypeptide may be recovered/isolated and optionally purified by any of a number of methods known in the art. For example, the lignocellulose degradation polypeptide may be isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. For example, purification of a glycohydrolase is described in US patent publication US 2007/0238155, incorporated herein by reference. In addition to the references noted supra, a variety of purification methods are well known in the art, including, for example, those set forth in Sandana (1997) Bioseparation of Proteins, Academic Press, Inc.; Bollag et al. (1996) Protein Methods, 2^{nd} Edition, Wiley-Liss, NY; Walker (1996) The Protein Protocols Handbook Humana Press, NJ; Harris and Angal (1990) Protein Purification Applications: A Practical Approach, IRL Press at Oxford, Oxford, England; Harris and Angal Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference.

Immunological methods may also be used to purify a lignocellulose degradation polypeptide. In one approach, an antibody raised against the enzyme using conventional methods is immobilized on beads, mixed with cell culture media under conditions in which the enzyme is bound, and precipitated. In a related approach immunochromatograpy is used. In some embodiments, purification is achieved using protein tags to isolate recombinantly expressed protein.

### VI. C1 Cells Having Absent or Decreased Expression of a Lignocellulose Degradation Enzyme

In a further aspect, the invention provides C1 cells in which expression of one or more lignocellulose degradation enzymes having a sequence set forth in Table 1 or Table 2 is inhibited. In the context of this invention, the term “inhibited” refers to a reduction in the level of the enzyme in an engineered C1 cell in which a nucleic acid sequence encoding a lignocellulose degradation enzyme has been targeted to decrease expression in comparison to wildtype cells. In typical embodiments, the genomic sequence expressing a target lignocellulose degradation enzyme of the invention is knocked out in C1 cells and expression of the enzyme is absent in the engineered cells.

Methods for introducing genetic mutations into C1 genes and selecting cells with reduced or absent expression of the protein of interest are well known. For instance, C1 can be treated with a mutagenic chemical substance, according to standard techniques. Such chemical substances include, but are not limited to, the following: NTG, diethyl sulfate, ethylene imine, ethyl methanesulfonate and N-nitroso-N-ethylurea. Alternatively, ionizing radiation from sources such as X-rays or gamma rays can be used, or non-ionizing UV radiation can be employed. In other embodiments, insertional or transposon mutagenesis can be performed.

Alternatively, homologous recombination can be used to induce targeted gene modifications by specifically targeting a lignocellulose degradation enzyme gene in vivo to suppress expression (see, generally, Grewal and Klar, Genetics 146: 1221-1238 (1997) and Xu et al., Genes Dev. 10: 2411-2422 (1996)). In applying homologous recombination technology to the genes of the invention, mutations in selected portions of a lignocellulose degradation enzyme gene sequences are made in vitro and then introduced into the C1 host using standard techniques. The mutated gene will interact with the target wild-type gene in such a way that homologous recombination and targeted replacement of the wild-type gene occurs in the host cells, resulting in suppression of activity of the protein encoded by the gene.

In other embodiments, insertional mutagenesis can be used to mutagenize a population of host cells that can subsequently be screened.

In some embodiments, the invention provides a transgenic C1 cell that is characterized by reduced lignocellulose degradation enzyme expression due to suppression of expression of a nucleic acid molecule encoding a lignocellulose degradation polypeptide. Such a cell may comprise an expression cassette stably transformed into the cell, such that that expression is inhibited constitutively or under certain conditions, e.g., when an inducible promoter is used.

A number of methods can be used to inhibit gene expression of a lignocellulose degradation enzyme of Table 1 or Table 2. For instance, siRNA, antisense, or ribozyme technology can be conveniently used that targets a nucleic acid sequence that encodes a lignocellulose degradation enzyme of Table 1 or Table 2. Such techniques are well known in the art. Thus, the invention further provides a sequence complementary to the nucleotide sequence of the lignocellulose enzyme gene that is capable of hybridizing to the mRNA produced in the cell to inhibit the amount of protein expressed.

C1 cells manipulated to inhibit expression of a lignocellulose degradation enzyme of the invention can be screened for decreased gene expression using standard assays to determine the levels of RNA and/or protein expression, which assays include quantitative RT-PCR, immunoassays and/or enzymatic activity assays. Such C1 cells can be used as host cells for the expression of native and/or heterologous polypeptides.

Thus, in a further aspect, the invention additionally provides a recombinant host cell comprising a disruption or deletion of a polynucleotide sequence identified in Table 1 or Table 2, e.g., Table 2, wherein the disruption or deletion inhibits expression of the lignocellulose degradation enzyme encoded by the polynucleotide sequence. In some embodiments, the recombinant host cell comprises an anti-sense RNA or iRNA that is complementary to a polynucleotide sequence identified in Table 1 or Table 2.

### VII. Methods of Using Lignocellulose Degration Enzymes and Cells Expressing the Enzymes

As described supra, lignocellulose degradation polypeptides of the present invention can be used to degrade cellulosic biomass, e.g., a glycoside hydrolase of Table 1 or Table 2 can be used to catalyze the hydrolysis of a sugar dimer with the release of the corresponding sugar monomer. In some embodiments, a lignocellulose degradation polypeptide of the invention participates in the degradation of cellulosic biomass to obtain a carbohydrate not by directly hydrolyzing cellulose or hemicellulose to obtain the carbohydrate, but by generating a degradation product that is more readily hydrolyzed to a carbohydrate by cellulases and accessory proteins. For example, lignin can be broken down using a lignocellulose degradation enzyme of the invention, such as a laccase, to provide an intermediate in which more cellulose or hemicellulose is accessible for degradation by cellulases and glycoside hydrolases. Various other enzymes, e.g., endoglucanases and cellobiohydrolases catalyze the hydrolysis of insoluble cellulose to cellooligosaccharides while β-glucosidases convert the oligosaccharides to glucose. Similarly, xylanases, together with other enzymes such as α-L-arabinofuranosidases, ferulic and acetylxylan esterases and β-xylosidases, catalyze the hydrolysis of hemicelluloses.

The present invention thus further provides compositions that are useful for the enzymatic conversion of a cellulosic biomass to soluble carbohydrates. For example, one or more lignocellulose degradation polypeptides of the present invention may be combined with one or more other enzymes and/or an agent that participates in lignocellulose degradation. The other enzyme(s) may be a different glycoside hydrolase or an accessory protein such as an esterase, oxidase, or the like; or an ortholog, e.g., from a different organism of an enzyme of the invention.

### Cellulosic Biomass Degradation Mixtures

For example, in some embodiments, a glycoside hydrolase lignocellulose degradation enzyme set forth in Table 1 or Table 2 may be combined with other glycoside hydrolases to form a mixture or composition comprising a recombinant lignocellulose degradation enzyme of the present invention and a C1 cellulase or other filamentous fungal cellulase. The mixture or composition may include cellulases selected from CBH, EG and BG cellulases (e.g., cellulases from a Trichoderma sp. (e.g. Trichoderma reesei and the like); an Acidothermus sp. (e.g., Acidothermus cellulolyticus, and the like); an Aspergillus sp. (e.g., Aspergillus nidulans, Aspergillus niger, Aspergillus oryzae, and the like); a Humicola sp. (e.g., Humicola grisea, and the like); a Chrysosporium sp., as well as cellulases derived from any of the host cells described under the section entitled “Expression Hosts”, supra).

The mixture may additionally comprise one or more accessory proteins, e.g., an accessory enzyme such as an esterase to de-esterify hemicellulose, set forth in Table 1 or Table 2; and/or accessory proteins from other organisms. The enzymes of the mixture work together resulting in hydrolysis of the hemicellulose and cellulose from a biomass substrate to yield soluble carbohydrates, such as, but not limited to, glucose and xylose (See Brigham et al., 1995, in Handbook on Bioethanol (C. Wyman ed.) pp 119-141, Taylor and Francis, Washington D.C., which is incorporated herein by reference). In some embodiments, mixtures of purified naturally occurring or recombinant enzymes are combined with cellulosic biomass or a product of lignocellulose hydrolysis. Alternatively or in addition, one or more cells producing naturally occurring or recombinant lignocellulose degradation enzymes may be used.

### Other Components of Enzyme Compositions

Lignocellulose degradation enzyme polypeptides of the present invention may be used in combination with other optional ingredients such as a buffer, a surfactant, and/or a scouring agent. A buffer may be used with an enzyme of the present invention (optionally combined with other cellulose degradation enzymes) to maintain a desired pH within the solution in which the enzyme is employed. The exact concentration of the buffer employed will depend on several factors which the skilled artisan can determine. Suitable buffers are well known in the art. A surfactant may further be used in combination with the enzymes of the present invention. Suitable surfactants include any surfactant compatible with the cellulose degradation enzyme of the invention and optional other enzymes being utilized. Exemplary surfactants include anionic, non-ionic, and ampholytic surfactants.

Production of Soluble Sugars from Cellulosic Biomass

Lignocellulose degradation enzymes of the present invention, as well as any composition, culture medium, or cell lysate comprising such polypeptides, may be used in the production of monosaccharides, disaccharides, or oligomers of a mono- or di-saccharide from biomass for subsequent use as chemical or fermentation feedstock or in chemical synthesis. As used herein, the term “cellulosic biomass” refers to living or dead biological material that contains a cellulose substrate, such as, for example, lignocellulose, hemicellulose, lignin, and the like. Therefore, the present invention provides a method of converting a biomass substrate to a degradation product, the method comprising contacting a culture medium or cell lysate containing a lignocellulose degradation polypeptide according to the invention, with the biomass substrate under conditions suitable for the production of the degradation product. The degradation product can be an end product such as a soluble sugar, or a product that undergoes further enzymatic conversion to an end product such as a soluble sugar. For example, a lignocellulose degradation enzyme of the invention may participate in a reaction that makes the cellulosic substrate more susceptible to hydrolysis so that the substrate is more readily hydrolyzed to fermentable sugars, such as glucose, cellobiose, xylose, xylulose, arabinose, mannose, galactose, and/or soluble oligosaccharides. The cellulosic substrate can be contacted with a composition, culture medium or cell lysate containing a lignocellulose degradation enzyme of Table 1 or Table 2 (and optionally other enzymes involved in breaking down cellulosic biomass) under conditions suitable for the production of a lignocellulose degradation product. In some embodiments, the contacting step may involve contacting the biomass with a composition, culture medium, or cell lysate containing an accessory protein such as an esterase, laccase, etc. set forth in Table 1 or Table 2. In some embodiments, the contacting step may involve contacting the biomass with a composition, culture medium, or cell lysate containing a glycosyl hydrolase set forth in Table 1 or Table 2.

Thus, the present invention provides a method for producing a lignocellulose degradation product by (a) providing a cellulosic biomass; and (b) contacting the biomass with at least one lignocellulose degradation enzyme that has an amino acid sequence set forth in Table 1 or Table 2 under conditions sufficient to form a reaction mixture for converting the biomass to a degradation product such as a soluble carbohydrate, or a product that is more readily hydrolyzed to a soluble carbohydrate. The cellulose degradation polypeptide may be used in such methods in either isolated form or as part of a composition, such as any of those described herein. The lignocellulose degradation enzyme may also be provided in cell culturing media or in a cell lysate. For example, after producing the lignocellulose degradation enzyme by culturing a host cell transformed with a lignocellulose degradation polynucleotide or vector of the present invention, the enzyme need not be isolated from the culture medium (i.e., if the enzyme is secreted into the culture medium) or cell lysate (i.e., if the enzyme is not secreted into the culture medium) or used in a purified form to be useful. Any composition, cell culture medium, or cell lysate containing a lignocellulose degradation enzyme of the present invention may be suitable for use in methods to degrade cellulosic biomass. Therefore, the present invention further provides a method for producing a degradation product of lignocellulose, such as a soluble sugar, a de-esterified cellulose biomass, etc. by: (a) providing a cellulosic biomass; and (b) contacting the biomass with a culture medium or cell lysate or composition comprising at least one lignocellulose degradation enzyme having an amino acid sequence of Table 1 or Table 2, e.g., a glycoside hydrolase of Table 1 or Table 2, under conditions sufficient to form a reaction mixture for converting the cellulosic biomass to the degradation product.

In some embodiments, the biomass includes cellulosic substrates including but not limited to, wood, wood pulp, paper pulp, corn stover, corn fiber, rice, paper and pulp processing waste, woody or herbaceous plants, fruit or vegetable pulp, distillers grain, grasses, rice hulls, wheat straw, cotton, hemp, flax, sisal, corn cobs, sugar cane bagasse, switch grass and mixtures thereof. The biomass may optionally be pretreated to increase the susceptibility of cellulose to hydrolysis using methods known in the art such as chemical, physical and biological pretreatments (e.g., steam explosion, pulping, grinding, acid hydrolysis, solvent exposure, and the like, as well as combinations thereof).

Soluble sugars produced by the methods of the present invention may be used to produce an alcohol (such as, for example, ethanol, butanol, and the like). The present invention therefore provides a method of producing an alcohol, where the method comprises (a) providing a soluble sugar produced using a lignocellulose degradation polypeptide of the present invention in the methods described supra; (b) contacting the soluble sugar with a fermenting microorganism to produce the alcohol or other metabolic product; and (c) recovering the alcohol or other metabolic product.

In some embodiments, the lignocellulose degradation polypeptide of the present invention, or composition, cell culture medium, or cell lysate containing the polypeptide, may be used to catalyze the hydrolysis of a biomass substrate to a soluble sugar in the presence of a fermenting microorganism such as a yeast (e.g., Saccharomyces sp., such as, for example, S. cerevisiae, Zymomonas sp., E. coli, Pichia sp., and the like) or other C5 or C6 fermenting microorganisms that are well known in the art, to produce an end-product such as ethanol. In this simultaneous saccharification and fermentation (SSF) process the soluble sugars (e.g., glucose and/or xylose) are removed from the system by the fermentation process.

The soluble sugars produced by the use of a lignocellulose degradation polypeptide of the present invention may also be used in the production of other end-products, such as, for example, acetone, an amino acid (e.g., glycine, lysine, and the like), an organic acid (e.g., lactic acid, and the like), glycerol, a diol (e.g., 1,3 propanediol, butanediol, and the like) and animal feeds.

One of skill in the art will readily appreciate that lignocellulose degradation polypeptide compositions of the present invention may be used in the form of an aqueous solution or a solid concentrate. When aqueous solutions are employed, the enzyme solution can easily be diluted to allow accurate concentrations. A concentrate can be in any form recognized in the art including, for example, liquids, emulsions, suspensions, gel, pastes, granules, powders, an agglomerate, a solid disk, as well as other forms that are well known in the art. Other materials can also be used with or included in the enzyme composition of the present invention as desired, including stones, pumice, fillers, solvents, enzyme activators, and anti-redeposition agents depending on the intended use of the composition.

The foregoing and other aspects of the invention may be better understood in connection with the following non-limiting examples.

### VIII. Examples

Tables 1 and 2 provide C1 lignocellulose degradation enzymes that were identified from the C1 genome sequence. The Pfam domains were identified using “PFAM v.24”, developed by the Wellcome Trust Sanger Institute, which is available at the web address “pfam.sanger.ac.uk/about” preceded by “http://”.

Various genes were selected for over-expression. The genes were cloned as genomic DNA fragments by PCR with flanking primers and cloned into an expression construct driven with the C1 chi1 promoter and cbh1a terminator. The constructs were transformed either into a C1 strain DC9 or a C1 strain DC18. A selection marker, typically Phleomycin, was used to select transformants. Transformants were fermented and the produced supernatant was analyzed with SDS-PAGE. The results showed that the various genes were over-expressed in the C1 strains. The over expressed genes were SEQ ID NO:127 (CBDH), SEQ ID NO:51 (arabinogalactanase), SEQ ID NO: 121 (ferulic acid esterase), SEQ ID NO:63 (endoarabinase), SEQ ID NO:167, SEQ ID NO:173 (CBM), SEQ ID NO: 177 (muc-lac enzyme), SEQ ID NO:447 (acetylxylan esterase), SEQ ID NO:25 (cbh), SEQ ID NO:575, and SEQ ID NO:321.

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

TABLE 1

| | | polypeptide | | | |
|---|---|---|---|---|---|
| nucleic acid | amino acid | length (no. | signal | | |
| seq no | seq id no. | amino acids) | peptide | Pfam domains | Functional class |
| 1 | 2 | 454 | YES | GH72-GH5(low score) | glycohydrolase |
| 3 | 4 | 460 | YES | GH72--GH5(low score) | glycohydrolase |
| 5 | 6 | 764 | NO | GH5 | glycohydrolase |
| 7 | 8 | 356 | YES | GH3 | glycohydrolase |
| 9 | 10 | 476 | NO | GH1--GH5 | glycohydrolase |
| 11 | 12 | 410 | YES | GH5--GH42--GH2_C | glycohydrolase |
| 13 | 14 | 760 | YES | GH3--GH3_C | glycohydrolase |
| 15 | 16 | 456 | YES | GH7 | glycohydrolase |
| 17 | 18 | 128 | YES | GH7 | glycohydrolase |
| 19 | 20 | 464 | YES | GH7--CBM_1 | glycohydrolase |
| 21 | 22 | 222 | YES | GH11 | glycohydrolase |
| 23 | 24 | 326 | YES | GH10 | glycohydrolase |
| 25 | 26 | 395 | YES | GH6 | glycohydrolase |
| 27 | 28 | 225 | YES | GH45 | glycohydrolase |
| 29 | 30 | 482 | YES | CBM_1--GH6 | glycohydrolase |
| 31 | 32 | 278 | YES | GH11--CBM_1 | glycohydrolase |
| 33 | 34 | 321 | YES | GH62 | glycohydrolase |
| 35 | 36 | 228 | YES | GH11 | glycohydrolase |
| 37 | 38 | 733 | YES | GH3--GH3_C | glycohydrolase |
| 39 | 40 | 247 | YES | GH12 | glycohydrolase |
| 41 | 42 | 680 | NO | GH15 | glycohydrolase |
| 43 | 44 | 225 | YES | GH25 | glycohydrolase |
| 45 | 46 | 405 | YES | GH17 | glycohydrolase |
| 47 | 48 | 628 | YES | GH76--GH76 | glycohydrolase |
| 49 | 50 | 351 | NO | GH18 | glycohydrolase |
| 51 | 52 | 350 | YES | GH53 | glycohydrolase |
| 53 | 54 | 280 | YES | GH16--SKN1 | glycohydrolase |
| 55 | 56 | 601 | YES | GH47 | glycohydrolase |
| 57 | 58 | 483 | YES | GH72 | glycohydrolase |
| 59 | 60 | 1052 | NO | GH63 | glycohydrolase |
| 61 | 62 | 669 | NO | GH31 | glycohydrolase |
| 63 | 64 | 321 | YES | GH43 | glycohydrolase |
| 65 | 66 | 254 | YES | Polysacc_deac_1--GH57 | glycohydrolase |
| 67 | 68 | 897 | NO | GH2_N--GH2--GH2_C | glycohydrolase |
| 69 | 70 | 392 | NO | DUF1680--GH88 | glycohydrolase |
| 71 | 72 | 285 | YES | GH16--SKN1 | glycohydrolase |
| 73 | 74 | 844 | YES | GH92 | glycohydrolase |
| 75 | 76 | 537 | NO | GH43 | glycohydrolase |
| 77 | 78 | 898 | | GH47 | glycohydrolase |
| 79 | 80 | 418 | YES | GH18 | glycohydrolase |
| 81 | 82 | 327 | NO | GH43--GH43 | glycohydrolase |
| 83 | 84 | 269 | YES | GH16--SKN1 | glycohydrolase |
| 85 | 86 | 558 | YES | GH16 | glycohydrolase |
| 87 | 88 | 426 | YES | GH18 | glycohydrolase |
| 89 | 90 | 451 | YES | GH43 | glycohydrolase |
| 91 | 92 | 518 | NO | AMP-binding--GH3 | glycohydrolase |
| 93 | 94 | 533 | YES | GH72--GH2_C--X8 | glycohydrolase |
| 95 | 96 | 606 | | GH47 | glycohydrolase |
| 97 | 98 | 454 | YES | GH76 | glycohydrolase |
| 99 | 100 | 403 | NO | GH18 | glycohydrolase |
| 101 | 102 | 358 | NO | GH18 | glycohydrolase |
| 103 | 104 | 586 | | Metallophos | accessroy protein |
| 105 | 106 | 683 | NO | Lipase_3 | accessory protein |
| 107 | 108 | 320 | | NAD_binding_2--3HCDH_N--DAO-- | accessory protein |
| | | | | Saccharop_dh--ApbA--3HCDH | |
| 109 | 110 | 423 | NO | Cutinase--Abhydrolase_1 | accessory protein |
| 111 | 112 | 383 | NO | GDPD | accessory protein |
| 113 | 114 | 505 | NO | Thi4--HI0933_like--Pyr_redox_2--Pyr_redox- | accessory protein |
| | | | | FAD_binding_2--DAO--GIDA--Pyr_redox-- | |
| | | | | 3HCDH_N--Pyr_redox_dim | |
| 115 | 116 | 441 | YES | HI0933_like--FAD_binding_2--Pyr_redox_2-- | accessory protein |
| | | | | DAO--Pyr_redox--GIDA--FAD_binding_3 | |
| 117 | 118 | 376 | NO | COesterase--Abhydrolase_3 | accessory protein |
| 119 | 120 | 225 | YES | DOMON | accessory protein |
| 121 | 122 | 279 | YES | Peptidase_S9--Esterase_phd-- | accessory protein |
| | | | | Abhydrolase_2--AXE1 | |
| 123 | 124 | 263 | YES | Lipase_GDSL | accessory protein |
| 125 | 126 | 238 | NO | FSH1--Abhydrolase_2--Thioesterase | accessory protein |
| 127 | 128 | 828 | YES | DOMON--GMC_oxred_N--HI0933_like-- | accessory protein |
| | | | | FAD_binding_2--DAO--Pyr_redox_2-- | |
| | | | | GMC_oxred_C--CBM_1 | |
| 129 | 130 | 911 | NO | PDEase_I | accessory protein |
| 131 | 132 | 292 | NO | Esterase--Esterase_phd--Peptidase_S9 | accessory protein |
| 133 | 134 | 355 | | peroxidase | accessory protein |
| 135 | 136 | 515 | NO | esterase | accessory protein |
| 137 | 138 | 588 | | Abhydrolase_1--Esterase | accessory protein |
| 139 | 140 | 565 | NO | Peptidase_C65 | accessory protein |
| 141 | 142 | 677 | NO | PI-PLC-X--PI-PLC-Y | accessory protein |
| 143 | 144 | 244 | | Abhydrolase_2--FSH1--DLH--Peptidase_S9 | accessory protein |
| 145 | 146 | 576 | YES | COesterase--Abhydrolase_3 | accessory protein |
| 147 | 148 | 623 | YES | GMC_oxred_N--GMC_oxred_C | accessory protein |
| 149 | 150 | 188 | NO | 4HBT | accessory protein |
| 151 | 152 | 231 | YES | Cutinase--PE-PPE | accessory protein |
| 153 | 154 | 650 | YES | laccase-like Cu-oxidase_3--Cu-oxidase--Cu- | accessory protein |
| | | | | oxidase_2 | |
| 155 | 156 | 212 | NO | | accessory protein |
| 157 | 158 | 235 | YES | Lipase_GDSL | accessory protein |
| 159 | 160 | 303 | NO | DUF1989 | accessory protein |
| 161 | 162 | 573 | NO | Tyr-DNA_phospho | accessory protein |
| 163 | 164 | 424 | YES | Tyrosinase | accessory protein |
| 165 | 166 | 443 | NO | FAD_binding_3--DAO--SE | accessory protein |
| 167 | 168 | 348 | YES | CBM_1 | accessory protein |
| 169 | 170 | 291 | YES | Esterase_phd--Peptidase_S9-- | accessory protein |
| | | | | Abhydrolase_1 | |
| 171 | 172 | 408 | NO | Beta-lactamase | accessory protein |
| 173 | 174 | 1076 | YES | | accessory protein |
| 175 | 176 | 220 | YES | WSC--WSC | accessory protein |
| 177 | 178 | 401 | YES | Muc_lac_enz | accessory protein |

TABLE 2

| | | | minimum | | | |
|---|---|---|---|---|---|---|
| | | polypeptide | fragment | | | |
| nucleic acid | amino acid | length (no. | size (no. | signal | | |
| seq id no | seq id no | amino acids) | amino acids) | peptide | Pfam domains; function | Functional class |
| 179 | 180 | 270 | | NO | GH3 | glycohydrolase |
| 181 | 182 | 491 | 421 | NO | GH5 | glycohydrolase |
| 183 | 184 | 506 | 414 | NO | GH10--CBM_1 | glycohydrolase |
| 185 | 186 | 226 | 214 | YES | GH11 | glycohydrolase |
| 187 | 188 | 69 | | YES | GH7 | glycohydrolase |
| 189 | 190 | 373 | | YES | GH10 | glycohydrolase |
| 191 | 192 | 285 | 231 | NO | GH11 | glycohydrolase |
| 193 | 194 | 147 | | NO | GH11 | glycohydrolase |
| 195 | 196 | 370 | | YES | GH62--CBM_1 | glycohydrolase |
| 197 | 198 | 180 | | NO | GH3_C | glycohydrolase |
| 199 | 200 | 856 | 491 | NO | Aminotran_1_2--GH43--GH43 | glycohydrolase |
| 201 | 202 | 185 | | YES | GH30 | glycohydrolase |
| 203 | 204 | 652 | 629 | | GH15--CBM_20 | glycohydrolase |
| 205 | 206 | 673 | | YES | GH67N--GH67M--GH67C | glycohydrolase |
| 207 | 208 | 904 | 219 | NO | GH18--LysM | glycohydrolase |
| 209 | 210 | 819 | 522 | NO | Rad10--GH47 | glycohydrolase |
| 211 | 212 | 215 | | NO | GH71 | glycohydrolase |
| 213 | 214 | 484 | 472 | YES | GH30 | glycohydrolase |
| 215 | 216 | 493 | 476 | NO | GH76 | glycohydrolase |
| 217 | 218 | 614 | | YES | GH31 | glycohydrolase |
| 219 | 220 | 410 | 350 | YES | GH16--PAAR_motif | glycohydrolase |
| 221 | 222 | 900 | 873 | YES | GH2_N--GH2--GH2_C--Big_1 | glycohydrolase |
| 223 | 224 | 391 | 375 | YES | GH18 | glycohydrolase |
| 225 | 226 | 248 | 219 | YES | GH16 | glycohydrolase |
| 227 | 228 | 417 | 400 | YES | GH76 | glycohydrolase |
| 229 | 230 | 343 | | NO | GH31 | glycohydrolase |
| 231 | 232 | 616 | 372 | NO | GMC_oxred_N--GMC_oxred_C; alcohol oxidase | accessroy protein |
| 233 | 234 | 87 | | NO | Thi4 | accessory protein |
| 235 | 236 | 535 | | NO | DUF2424--Abhydrolase_3--Abhydrolase_3; esterase | accessory protein |
| 237 | 238 | 130 | | NO | esterase | accessory protein |
| 239 | 240 | 345 | | NO | Abhydrolase_2--Peptidase_S9--Abhydrolase_2; | accessory protein |
| | | | | | esterase | |
| 241 | 242 | 395 | | NO | esterase | accessory protein |
| 243 | 244 | 77 | | YES | esterase | accessory protein |
| 245 | 246 | 1155 | | NO | FAD_binding_2--Pyr_redox_2--GMC_oxred_N-- | accessory protein |
| | | | | | GMC_oxred_C | |
| 247 | 248 | 423 | 424 | YES | laccase-like_Cu-oxidase_2--Cu-oxidase_3 | accessory protein |
| 249 | 250 | 121 | 52 | NO | esterase | accessory protein |
| 251 | 252 | 137 | | NO | DUF676--PGAP1; esterase associate Pfam domain | accessory protein |
| 253 | 254 | 211 | | NO | Thi4--DAO--Pyr_redox_2--Lycopene_cycl-- | accessory protein |
| | | | | | FAD_binding_2 | |
| 255 | 256 | 425 | 346 | NO | Thi4--FAD_binding_3--HI0933_like--Lycopene_cycl-- | accessory protein |
| | | | | | DAO--Pyr_redox_2--FAD_binding_2 | |
| 257 | 258 | 337 | | NO | Hydrolase_4--Abhydrolase_1 | accessory protein |
| 259 | 260 | 1398 | 719 | NO | Cu_amine_oxidN2--Cu_amine_oxidN3-- | accessory protein |
| | | | | | Cu_amine_oxid--Cu_amine_oxid--Fungal_trans | |
| 261 | 262 | 645 | 624 | NO | PDEase_II--PDEase_II--PDEase_II | accessory protein |
| 263 | 264 | 271 | | NO | Lipase_GDSL | accessory protein |
| 265 | 266 | 387 | | NO | esterase | accessory protein |
| 267 | 268 | 260 | | YES | DUF2424--Abhydrolase_3--Peptidase_S9; esterase- | accessory protein |
| | | | | | lipase | |
| 269 | 270 | 103 | | YES | esterase-lipase | accessory protein |
| 271 | 272 | 805 | | YES | WSC--WSC--WSC--WSC--Glyoxal_oxid_N | accessory protein |
| 273 | 274 | 831 | 314 | NO | FMN_red--Flavodoxin_2--Flavodoxin_1--PUA-- | accessory protein |
| | | | | | PNP_UDP_1 | |
| 275 | 276 | 232 | 227 | YES | Lipase_GDSL | accessory protein |
| 277 | 278 | 483 | | NO | laccase-like Cu-oxidase_3--Cu-oxidase | accessory protein |
| 279 | 280 | 110 | | NO | laccase-like Cu-oxidase_2 | accessory protein |
| 281 | 282 | 54 | | | esterase | accessory protein |
| 283 | 284 | 644 | 615 | NO | Exo_endo_phos--zf-GRF | accessory protein |
| 285 | 286 | 392 | 336 | YES | Tyrosinase | accessory protein |
| 287 | 288 | 122 | | NO | Acyl-ACP_TE--4HBT | accessory protein |
| 289 | 290 | 303 | 85 | YES | | accessory protein |
| 291 | 292 | 709 | | YES | DOMON--Thi4--GMC_oxred_N--HI0933_like-- | accessory protein |
| | | | | | FAD_binding_2--DAO--Pyr_redox_2; alcohol oxidase | |
| | | | | | homolog | |
| 293 | 294 | 390 | 337 | YES | Tyrosinase | accessory protein |
| 295 | 296 | 498 | | NO | FAD_binding_3 | accessory protein |
| 297 | 298 | 332 | | YES | Palm_thioest | accessory protein |
| 299 | 300 | 641 | 363 | NO | Abhydrolase_2--LIP--Gln-synt_N--Gln-synt_C | accessory protein |
| 301 | 302 | 1210 | 1054 | NO | DUF676--BSP_II--DUF676 | accessory protein |
| 303 | 304 | 197 | | NO | esterase | accessory protein |
| 305 | 306 | 469 | | NO | Pyr_redox_2--DAO--GIDA--Pyr_redox-- | accessory protein |
| | | | | | Pyr_redox_dim | |
| 307 | 308 | 326 | 304 | YES | CBM_1 | accessory protein |
| 309 | 310 | 1543 | 928 | NO | GH3--GH3_C--NDT80_PhoG | glycohydrolase |
| 311 | 312 | 777 | | YES | GH3--GH3_C | glycohydrolase |
| 313 | 314 | 890 | | YES | GH3--GH3_C | glycohydrolase |
| 315 | 316 | 103 | | NO | GH3 | glycohydrolase |
| 317 | 318 | 968 | | NO | GH3--GH3_C | glycohydrolase |
| 319 | 320 | 827 | 810 | YES | GH3--GH3_C | glycohydrolase |
| 321 | 322 | 342 | 338 | YES | GH5 | glycohydrolase |
| 323 | 324 | 370 | | YES | GH5--GH2_C | glycohydrolase |
| 325 | 326 | 115 | | NO | GH10 | glycohydrolase |
| 327 | 328 | 64 | | NO | GH11 | glycohydrolase |
| 329 | 330 | 218 | | YES | GH11 | glycohydrolase |
| 331 | 332 | 83 | | YES | GH11 | glycohydrolase |
| 333 | 334 | 519 | | YES | GH7--CBM_1 | glycohydrolase |
| 335 | 336 | 867 | | NO | GH3--GH3_C | glycohydrolase |
| 337 | 338 | 398 | 327 | NO | GH10 | glycohydrolase |
| 339 | 340 | 381 | 359 | YES | GH6 | glycohydrolase |
| 341 | 342 | 1097 | 661 | YES | FAD_binding_3--Pyr_redox_2--GMC_oxred_N-- | glycohydrolase |
| | | | | | GMC_oxred_C--GH7 | |
| 343 | 344 | 395 | | YES | GH16 | glycohydrolase |
| 345 | 346 | 605 | 603 | YES | GH47 | glycohydrolase |
| 347 | 348 | 858 | 856 | NO | GH2_N--GH2--GH2_C | glycohydrolase |
| 349 | 350 | 304 | | YES | GH16 | glycohydrolase |
| 351 | 352 | 808 | 759 | NO | GH47 | glycohydrolase |
| 353 | 354 | 1080 | 1063 | NO | GH2_N--GH2--GH2_C--Bgal_small_N | glycohydrolase |
| 355 | 356 | 412 | | NO | GH20 | glycohydrolase |
| 357 | 358 | 416 | | NO | GH16 | glycohydrolase |
| 359 | 360 | 1113 | | NO | GH47 | glycohydrolase |
| 361 | 362 | 1084 | 1064 | NO | GH38--Alpha-mann_mid--GH38C | glycohydrolase |
| 363 | 364 | 374 | | NO | GH3_C--PA14 | glycohydrolase |
| 365 | 366 | 1468 | | YES | LysM--LysM--Chitin_bind_1--GH18 | glycohydrolase |
| 367 | 368 | 812 | | YES | GH92 | glycohydrolase |
| 369 | 370 | 648 | 577 | NO | GH47 | glycohydrolase |
| 371 | 372 | 812 | | YES | GH92 | glycohydrolase |
| 373 | 374 | 180 | | YES | GH43 | glycohydrolase |
| 375 | 376 | 387 | 360 | YES | GH16 | glycohydrolase |
| 377 | 378 | 320 | 292 | YES | GH43 | glycohydrolase |
| 379 | 380 | 611 | 587 | YES | GH43 | glycohydrolase |
| 381 | 382 | 542 | 490 | YES | GH43--GH43 | glycohydrolase |
| 383 | 384 | 475 | 364 | NO | GH76 | glycohydrolase |
| 385 | 386 | 397 | | YES | GH2_N--GH2 | glycohydrolase |
| 387 | 388 | 284 | | YES | GH43 | glycohydrolase |
| 389 | 390 | 638 | | NO | GH31--Raffinose_syn--Melibiase--Raffinose_syn | glycohydrolase |
| 391 | 392 | 810 | 787 | YES | GH81 | glycohydrolase |
| 393 | 394 | 456 | | YES | GH26--GH26 | glycohydrolase |
| 395 | 396 | 982 | 961 | YES | GH31 | glycohydrolase |
| 397 | 398 | 1134 | 791 | NO | LysM--Chitin_bind_1--GH18--DUF3142 | glycohydrolase |
| 399 | 400 | 435 | | YES | GH71 | glycohydrolase |
| 401 | 402 | 456 | | YES | GH76 | glycohydrolase |
| 403 | 404 | 534 | 522 | NO | GH18 | glycohydrolase |
| 405 | 406 | 393 | | YES | GH76--GH76 | glycohydrolase |
| 407 | 408 | 972 | | YES | GH31 | glycohydrolase |
| 409 | 410 | 870 | 764 | YES | GH2_N--GH2--GH2_C--GH2_C | glycohydrolase |
| 411 | 412 | 336 | | NO | GH43--GH43--AbfB | glycohydrolase |
| 413 | 414 | 386 | | NO | GH16 | glycohydrolase |
| 415 | 416 | 821 | 767 | NO | GH31 | glycohydrolase |
| 417 | 418 | 719 | 475 | YES | GH76 | glycohydrolase |
| 419 | 420 | 286 | 277 | YES | GH17 | glycohydrolase |
| 421 | 422 | 695 | 486 | NO | Zn_clus--GH16 | glycohydrolase |
| 423 | 424 | 416 | | YES | GH16 | glycohydrolase |
| 425 | 426 | 268 | | NO | GH2_N--GH2 | glycohydrolase |
| 427 | 428 | 629 | 590 | YES | GH71 | glycohydrolase |
| 429 | 430 | 850 | | NO | GH31 | glycohydrolase |
| 431 | 432 | 464 | | YES | GH76 | glycohydrolase |
| 433 | 434 | 838 | | YES | GH63--Trehalase | glycohydrolase |
| 435 | 436 | 423 | 402 | YES | GH28 | glycohydrolase |
| 437 | 438 | 1754 | 793 | NO | F-box--GH35--MFS_1--Sugar_tr | glycohydrolase |
| 439 | 440 | 878 | | YES | GH67N--GH67M--GH67C | glycohydrolase |
| 441 | 442 | 463 | 436 | YES | GH28 | glycohydrolase |
| 443 | 444 | 502 | 280 | NO | GH43 | glycohydrolase |
| 445 | 446 | 526 | 523 | YES | p450 | accessory protein |
| 447 | 448 | 213 | | YES | Cutinase--Abhydrolase_1 | accessory protein |
| 449 | 450 | 655 | 587 | YES | GMC_oxred_N--DAO--GMC_oxred_C | accessory protein |
| 451 | 452 | 578 | 525 | YES | Thi4--GMC_oxred_N--HI0933_like--DAO-- | accessory protein |
| | | | | | FAD_binding_2--Pyr_redox_2--FAD_binding_2--DAO-- | |
| | | | | | GMC_oxred_C | |
| 453 | 454 | 441 | 409 | YES | FAD_binding_3--HI0933_like--FAD_binding_2--DAO-- | accessory protein |
| | | | | | Pyr_redox_2--GIDA--Pyr_redox--FA_binding_3 | |
| 455 | 456 | 203 | 178 | YES | Cupin_5; oxidase domain | accessory protein |
| 457 | 458 | 173 | | YES | esterase | accessory protein |
| 459 | 460 | 251 | | YES | Peptidase_S9--Abhydrolase_1--Esterase_phd | accessory protein |
| 461 | 462 | 265 | | NO | Pex14_N--SR-25--4HBT | accessory protein |
| 463 | 464 | 602 | 506 | NO | FAD_binding_3--DAO | accessory protein |
| 465 | 466 | 283 | | NO | Thioesterase | accessory protein |
| 467 | 468 | 582 | 503 | NO | Erythro_esteras--Erythro_esteras | accessory protein |
| 469 | 470 | 437 | | YES | Lipase_GDSL | accessory protein |
| 471 | 472 | 533 | 508 | | p450 | accessory protein |
| 473 | 474 | 318 | 311 | | PGAP1--Thioesterase--Abhydrolase_1--Esterase | accessory protein |
| 475 | 476 | 670 | 582 | NO | DUF676--PGAP1--UPF0227 | accessory protein |
| 477 | 478 | 127 | | NO | Lipase_3 | accessory protein |
| 479 | 480 | 860 | | YES | peroxidase--WSC--WSC | accessory protein |
| 481 | 482 | 1128 | | NO | efhand_like--PI-PLC-X--PI-PLC-Y--C2--esterase | accessory protein |
| 483 | 484 | 132 | | NO | DUF2343 | accessory protein |
| 485 | 486 | 141 | | YES | esterase | accessory protein |
| 487 | 488 | 418 | | YES | HI0933_like--DAO--FAD_binding_2--Pyr_redox_2-- | accessory protein |
| | | | | | Lycopene_cycl--DAO--FAD_binding_3 | |
| 489 | 490 | 221 | | NO | esterase | accessory protein |
| 491 | 492 | 2275 | 2237 | NO | ketoacyl-synt--Thiolase_N--Ketoacyl-synt_C-- | accessory protein |
| | | | | | Acyl_transf_1--PP-binding--PP-binding--Thioesterase | |
| | | | | | laccase-like Cu-oxidase_3--Cu-oxidase--Cu- | |
| 493 | 494 | 1287 | 735 | NO | oxidase_2 | accessory protein |
| 495 | 496 | 529 | | NO | esterase | accessory protein |
| 497 | 498 | 193 | | YES | Lipase_GDSL | accessory protein |
| 499 | 500 | 432 | | NO | COesterase--Abhydrolase_3; esterase-lipase | accessory protein |
| 501 | 502 | 1338 | | NO | Acyl_transf_1--PP-binding--PP-binding--Thioesterase-- | accessory protein |
| | | | | | Abhydrolase_1 | |
| 503 | 504 | 273 | | NO | COesterase--Abhydrolase_3 | accessory protein |
| 505 | 506 | 559 | 448 | | COesterase | accessory protein |
| 507 | 508 | 405 | 371 | YES | Tyrosinase | accessory protein |
| 509 | 510 | 769 | 742 | NO | GMC_oxred_N--GMC_oxred_C | accessory protein |
| 511 | 512 | 397 | | YES | DUF463--esterase | accessory protein |
| 513 | 514 | 394 | 335 | YES | Abhydrolase_2--Abhydrolase_3--Abhydrolase_1; | accessory protein |
| | | | | | esterase | |
| 515 | 516 | 1444 | | NO | DUF676--PGAP1--NB-ARC; esterase | accessory protein |
| 517 | 518 | 709 | 708 | NO | Cu_amine_oxidN2--Cu_amine_oxid | accessory protein |
| 519 | 520 | 254 | 221 | NO | laccase-like Cu-oxidase_3--Cu-oxidase--Cu- | accessory protein |
| | | | | | oxidase_2 | |
| 521 | 522 | 321 | 192 | NO | GMC_oxred_C | accessory protein |
| 523 | 524 | 416 | 394 | YES | Tannase--Tannase--Tannase | accessory protein |
| 525 | 526 | 592 | | NO | GMC_oxred_N--DAO--FAD_binding_2--Pyr_redox_2-- | accessory protein |
| | | | | | Lycopene_cycl--GMC_oxred_C; alcohol oxidase | |
| 527 | 528 | 205 | | NO | FOLy_LDA1_HMM | accessory protein |
| 529 | 530 | 383 | 351 | YES | Tyrosinase | accessory protein |
| 531 | 532 | 203 | | NO | esterase | accessory protein |
| 533 | 534 | 279 | | YES | Cutinase--PE-PPE--Cutinase--CBM_1; esterase | accessory protein |
| 535 | 536 | 409 | 347 | YES | Cupin_1--Cupin_2--Cupin_1--Cupin_3--Cupin_2; | accessory protein |
| | | | | | aldehyde oxidase | |
| 537 | 538 | 112 | | YES | esterase | accessory protein |
| 539 | 540 | 594 | | YES | p450 | accessory protein |
| 541 | 542 | 530 | | NO | esterase | accessory protein |
| 543 | 544 | 186 | | YES | GMC_oxred_N--GMC_oxred_C | accessory protein |
| 545 | 546 | 234 | | NO | DUF1749-esterase | accessory protein |
| 547 | 548 | 647 | 621 | YES | GMC_oxred_N--DAO--Lycopene_cycl-- | accessory protein |
| | | | | | GMC_oxred_C; alcohol oxidase | |
| 549 | 550 | 591 | | | Metallophos; esterase | accessory protein |
| 551 | 552 | 417 | | | esterase | accessory protein |
| 553 | 554 | 247 | | NO | Beta-lactamase; esterase | accessory protein |
| 555 | 556 | 1114 | | NO | Lipase_3 | accessory protein |
| 557 | 558 | 426 | | NO | DUF1100--Esterase--UPF0227--Esterase_phd-- | accessory protein |
| | | | | | Peptidase_S9 | |
| 559 | 560 | 231 | 231 | NO | | accessory protein |
| 561 | 562 | 638 | | NO | GMC_oxred_N--GMC_oxred_C alcohol oxidase | accessory protein |
| | | | | | domain | |
| 563 | 564 | 169 | | NO | esterase | accessory protein |
| 565 | 566 | 639 | | YES | laccase-like Cu-oxidase_3--Cu-oxidase_2--Cu- | accessory protein |
| | | | | | oxidase--Cu-oxidase_2 | |
| 567 | 568 | 316 | 271 | YES | Pectinesterase--Pectinesterase | accessory protein |
| 569 | 570 | 285 | | NO | FSH1--Abhydrolase_2--FSH1 | accessory protein |
| 571 | 572 | 219 | | NO | Esterase | accessory protein |
| 573 | 574 | 243 | | | Abhydrolase_3--Peptidase_S9--AXE1 | accessory protein |
| 575 | 576 | 313 | | YES | Esterase--Esterase_phd--Peptidase_S9--COesterase | accessory protein |
| 577 | 578 | 91 | | NO | CBM_1 | accessory protein |
| 579 | 580 | 682 | 665 | YES | COesterase--Abhydrolase_3 | accessory protein |
| 581 | 582 | 404 | | NO | DUF676--PGAP1 esterase domain | accessory protein |
| 583 | 584 | 227 | | YES | | accessory protein |
| 585 | 586 | 480 | | NO | PI-PLC-X--SR-25--PI-PLC-Y | accessory protein |
| 587 | 588 | 662 | 575 | NO | Tyr-DNA_phospho | accessory protein |
| 589 | 590 | 639 | | YES | Lipase_3 | accessory protein |
| 591 | 592 | 154 | 154 | YES | COesterase | accessory protein |
| 593 | 594 | 556 | | YES | COesterase--COesterase | accessory protein |
| 595 | 596 | 427 | 421 | YES | Tyrosinase | accessory protein |
| 597 | 598 | 602 | 602 | NO | Thi4--FAD_binding_3--DAO--FAD_binding_2-- | accessory protein |
| | | | | | Phe_hydrox_dim | |
| 599 | 600 | 474 | | NO | peroxidase | accessory protein |
| 601 | 602 | 305 | | NO | esterase | accessory protein |
| 603 | 604 | 866 | 857 | NO | DUF726--Thioesterase--DUF605 esterase domain | accessory protien |
| 605 | 606 | 644 | | YES | Thi4--FAD_binding_3--FAD_binding_2--Pyr_redox_2-- | accessory protein |
| | | | | | GIDA--DAO--Succ_DH_flav_C | |
| 607 | 608 | 292 | | NO | Lipase_3 | accessory protein |
| 609 | 610 | 645 | | YES | Tyrosinase | accessory protein |
| 611 | 612 | 502 | | YES | Cupin_1--3-HAO--Cupin_2--Cupin_1--Cupin_2 | accessory protein |
| | | | | | oxidase domain | |
| 613 | 614 | 451 | 427 | NO | Beta-lactamase | accessory protein |
| 615 | 616 | 483 | 473 | NO | esterase | accessory protein |
| 617 | 618 | 419 | 348 | NO | GMC_oxred_C | accessroy protein |
| 619 | 620 | 386 | | YES | Tyrosinase--GMC_oxred_N--GMC_oxred_C | accessory protein |
| 621 | 622 | 506 | 481 | NO | AP_endonuc_2 | accessory protein |
| 623 | 624 | 301 | 288 | | 4HBT | accessory protein |
| 625 | 626 | 693 | 555 | NO | COesterase--Abhydrolase_3 esterase-lipase domain | accessroy protein |
| 627 | 628 | 470 | 445 | YES | Cupin_1--Cupin_2--AraC_binding--Cupin_3--Cupin_1-- | accessory protein |
| | | | | | Cupin_3--Cupin_2 oxidase domain | |
| 629 | 630 | 217 | 201 | YES | Cupin_1--Cupin_2 | accessory protein |
| 631 | 632 | 375 | 364 | YES | Tyrosinase | accessory protein |
| 633 | 634 | 367 | 364 | NO | Acyl_CoA_thio--Acyl_CoA_thio | acceossry protein |
| 635 | 636 | 759 | 743 | NO | Phosphodiest | accessory protein |
| 637 | 638 | 404 | | YES | Lipase_GDSL | accessory protein |
| 639 | 640 | 627 | | NO | Abhydro_lipase--Abhydrolase_1 | accessory protein |
| 641 | 642 | 616 | | YES | laccase-like Cu-oxidase_3--Cu-oxidase--Cu- | accessory protein |
| | | | | | oxidase_2 domain | |
| 643 | 644 | 660 | 532 | NO | p450--p450 | accessory protein |
| 645 | 646 | 208 | | NO | COesterase--Abhydrolase_3 esterase domain | accessory protein |
| 647 | 648 | 295 | | NO | DUF2424--Abhydrolase_3 | accessory protein |
| 649 | 650 | 649 | | YES | p450 | accessory protein |
| 651 | 652 | 591 | 141 | NO | Retrotrans_gag--COesterase--Abhydrolase_3 | accessory protein |
| | | | | | esterase domain | |
| 653 | 654 | 564 | | NO | GMC_oxred_N--DAO--Lycopene_cycl--GMC_oxred_C | accessory protein |
| | | | | | alcohol oxidase domains | |
| 655 | 656 | 437 | 422 | YES | Glycos_transf_1 | accessory protein |
| 657 | 658 | 483 | | NO | DUF2424--Abhydrolase_3 | accessory protein |
| 659 | 660 | 624 | 624 | YES | PhoD | accessory protein |
| 661 | 662 | 1243 | 1124 | NO | DUF676--CorA esterase domain | accessory protein |
| 663 | 664 | 362 | 344 | YES | peroxidase | accessory protein |
| 665 | 666 | 168 | 153 | NO | 4HBT | accessory protein |
| 667 | 668 | 241 | | NO | CPDase | accessory protein |
| 669 | 670 | 301 | | NO | DUF676--PGAP1--LACT--Abhydrolase_1 | accessory protein |
| 671 | 672 | 505 | 448 | NO | SPX--SPX | accessory protein |
| 673 | 674 | 763 | 517 | NO | COesterase--Abhydrolase_3--Coesterase; esterase- | accessory protein |
| | | | | | lipase | |
| 675 | 676 | 230 | | NO | Abhydrolase_3 | accessory protein |
| 677 | 678 | 500 | | YES | p450--p450 | accessory protein |
| 679 | 680 | 785 | 377 | NO | GDPD--SET | accessory protein |
| 681 | 682 | 524 | | NO | Thioesterase--Abhydrolase_1--DUF915--Esterase | accessory protein |
| 683 | 684 | 295 | 269 | YES | Lipase_GDSL | accessory protein |
| 685 | 686 | 793 | | NO | RNA_lig_T4_1--tRNA_lig_kinase--tRNA_lig_CPD | accessory protein |
| 687 | 688 | 1505 | | NO | cNMP_binding--cNMP_binding--Patatin | accessory protein |
| 689 | 690 | 169 | | YES | Metallophos | accessory protein |
| 691 | 692 | 299 | | NO | Abhydrolase_2--FSH1--Abhydrolase_3-- | accessory protein |
| | | | | | Abhydrolase_2 | |
| 693 | 694 | 236 | | YES | CHAP | accessory protein |
| 695 | 696 | 202 | | YES | Tyrosinase | accessory protein |
| 697 | 698 | 649 | | NO | FAD_binding_3--Pyr_redox--Phe_hydrox_dim | accessory protein |
| 699 | 700 | 239 | | YES | Esterase_phd | accessory protein |
| 701 | 702 | 1201 | 1163 | NO | SPX--SPX--Ank--Ank--GDPD | accessory protein |
| 703 | 704 | 718 | 697 | YES | COesterase--Abhydrolase_3; esterase lipase | accessory protein |
| 705 | 706 | 386 | | YES | Phosphoesterase | accessory protein |
| 707 | 708 | 111 | | NO | 4HBT | accessory protein |
| 709 | 710 | 380 | 374 | YES | Dioxygenase_C | accessory protein |
| 711 | 712 | 582 | | YES | WSC--WSC | accessory protein |
| 713 | 714 | 906 | 540 | YES | RhgB_N--Peptidase_M28--Peptidase_M20 | accessory protein |
| 715 | 716 | 336 | | YES | Dioxygenase_C | accessory protein |
| 717 | 718 | 225 | | YES | Tannase--Tannase | accessory protein |
| 719 | 720 | 66 | | NO | esterase | accessory protein |

1. A recombinant Myceliophthera thermophilus lignocellulose degradation enzyme of Table 1 or Table 2, wherein the enzyme is selected from the group consisting of a glycohydrolase, an esterase, an oxidase, and an oxidoreductase; or wherein the enzyme comprises an amino acid sequence selected from the group consisting of SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, and SEQ ID NO: 720.

2. The recombinant lignocellulose degradation enzyme of claim 1, wherein the enzyme is a glycohydrolase.

3. (canceled)

4. An isolated nucleic acid encoding a polypeptide of claim 1.

5. The isolated nucleic acid of claim 4, wherein the nucleic acid has a sequence selected from the group consisting of SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, and SEQ ID NO: 719.

6. A recombinant vector comprising an isolated nucleic acid of claim 4, wherein the nucleic acid is operably linked to a promoter.

7. The recombinant vector of claim 6, wherein the promoter is a heterologous promoter.

8. A recombinant vector comprising a nucleic acid encoding a polypeptide having a sequence selected from the group consisting of SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 198, SEQ ID NO: 200, SEQ ID NO: 202, SEQ ID NO: 204, SEQ ID NO: 206, SEQ ID NO: 208, SEQ ID NO: 210, SEQ ID NO: 212, SEQ ID NO: 214, SEQ ID NO: 216, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, SEQ ID NO: 286, SEQ ID NO: 288, SEQ ID NO: 290, SEQ ID NO: 292, SEQ ID NO: 294, SEQ ID NO: 296, SEQ ID NO: 298, SEQ ID NO: 300, SEQ ID NO: 302, SEQ ID NO: 304, SEQ ID NO: 306, SEQ ID NO: 308, SEQ ID NO: 310, SEQ ID NO: 312, SEQ ID NO: 314, SEQ ID NO: 316, SEQ ID NO: 318, SEQ ID NO: 320, SEQ ID NO: 322, SEQ ID NO: 324, SEQ ID NO: 326, SEQ ID NO: 328, SEQ ID NO: 330, SEQ ID NO: 332, SEQ ID NO: 334, SEQ ID NO: 336, SEQ ID NO: 338, SEQ ID NO: 340, SEQ ID NO: 342, SEQ ID NO: 344, SEQ ID NO: 346, SEQ ID NO: 348, SEQ ID NO: 350, SEQ ID NO: 352, SEQ ID NO: 354, SEQ ID NO: 356, SEQ ID NO: 358, SEQ ID NO: 360, SEQ ID NO: 362, SEQ ID NO: 364, SEQ ID NO: 366, SEQ ID NO: 368, SEQ ID NO: 370, SEQ ID NO: 372, SEQ ID NO: 374, SEQ ID NO: 376, SEQ ID NO: 378, SEQ ID NO: 380, SEQ ID NO: 382, SEQ ID NO: 384, SEQ ID NO: 386, SEQ ID NO: 388, SEQ ID NO: 390, SEQ ID NO: 392, SEQ ID NO: 394, SEQ ID NO: 396, SEQ ID NO: 398, SEQ ID NO: 400, SEQ ID NO: 402, SEQ ID NO: 404, SEQ ID NO: 406, SEQ ID NO: 408, SEQ ID NO: 410, SEQ ID NO: 412, SEQ ID NO: 414, SEQ ID NO: 416, SEQ ID NO: 418, SEQ ID NO: 420, SEQ ID NO: 422, SEQ ID NO: 424, SEQ ID NO: 426, SEQ ID NO: 428, SEQ ID NO: 430, SEQ ID NO: 432, SEQ ID NO: 434, SEQ ID NO: 436, SEQ ID NO: 438, SEQ ID NO: 440, SEQ ID NO: 442, SEQ ID NO: 444, SEQ ID NO: 446, SEQ ID NO: 448, SEQ ID NO: 450, SEQ ID NO: 452, SEQ ID NO: 454, SEQ ID NO: 456, SEQ ID NO: 458, SEQ ID NO: 460, SEQ ID NO: 462, SEQ ID NO: 464, SEQ ID NO: 466, SEQ ID NO: 468, SEQ ID NO: 470, SEQ ID NO: 472, SEQ ID NO: 474, SEQ ID NO: 476, SEQ ID NO: 478, SEQ ID NO: 480, SEQ ID NO: 482, SEQ ID NO: 484, SEQ ID NO: 486, SEQ ID NO: 488, SEQ ID NO: 490, SEQ ID NO: 492, SEQ ID NO: 494, SEQ ID NO: 496, SEQ ID NO: 498, SEQ ID NO: 500, SEQ ID NO: 502, SEQ ID NO: 504, SEQ ID NO: 506, SEQ ID NO: 508, SEQ ID NO: 510, SEQ ID NO: 512, SEQ ID NO: 514, SEQ ID NO: 516, SEQ ID NO: 518, SEQ ID NO: 520, SEQ ID NO: 522, SEQ ID NO: 524, SEQ ID NO: 526, SEQ ID NO: 528, SEQ ID NO: 530, SEQ ID NO: 532, SEQ ID NO: 534, SEQ ID NO: 536, SEQ ID NO: 538, SEQ ID NO: 540, SEQ ID NO: 542, SEQ ID NO: 544, SEQ ID NO: 546, SEQ ID NO: 548, SEQ ID NO: 550, SEQ ID NO: 552, SEQ ID NO: 554, SEQ ID NO: 556, SEQ ID NO: 558, SEQ ID NO: 560, SEQ ID NO: 562, SEQ ID NO: 564, SEQ ID NO: 566, SEQ ID NO: 568, SEQ ID NO: 570, SEQ ID NO: 572, SEQ ID NO: 574, SEQ ID NO: 576, SEQ ID NO: 578, SEQ ID NO: 580, SEQ ID NO: 582, SEQ ID NO: 584, SEQ ID NO: 586, SEQ ID NO: 588, SEQ ID NO: 590, SEQ ID NO: 592, SEQ ID NO: 594, SEQ ID NO: 596, SEQ ID NO: 598, SEQ ID NO: 600, SEQ ID NO: 602, SEQ ID NO: 604, SEQ ID NO: 606, SEQ ID NO: 608, SEQ ID NO: 610, SEQ ID NO: 612, SEQ ID NO: 614, SEQ ID NO: 616, SEQ ID NO: 618, SEQ ID NO: 620, SEQ ID NO: 622, SEQ ID NO: 624, SEQ ID NO: 626, SEQ ID NO: 628, SEQ ID NO: 630, SEQ ID NO: 632, SEQ ID NO: 634, SEQ ID NO: 636, SEQ ID NO: 638, SEQ ID NO: 640, SEQ ID NO: 642, SEQ ID NO: 644, SEQ ID NO: 646, SEQ ID NO: 648, SEQ ID NO: 650, SEQ ID NO: 652, SEQ ID NO: 654, SEQ ID NO: 656, SEQ ID NO: 658, SEQ ID NO: 660, SEQ ID NO: 662, SEQ ID NO: 664, SEQ ID NO: 666, SEQ ID NO: 668, SEQ ID NO: 670, SEQ ID NO: 672, SEQ ID NO: 674, SEQ ID NO: 676, SEQ ID NO: 678, SEQ ID NO: 680, SEQ ID NO: 682, SEQ ID NO: 684, SEQ ID NO: 686, SEQ ID NO: 688, SEQ ID NO: 690, SEQ ID NO: 692, SEQ ID NO: 694, SEQ ID NO: 696, SEQ ID NO: 698, SEQ ID NO: 700, SEQ ID NO: 702, SEQ ID NO: 704, SEQ ID NO: 706, SEQ ID NO: 708, SEQ ID NO: 710, SEQ ID NO: 712, SEQ ID NO: 714, SEQ ID NO: 716, SEQ ID NO: 718, and SEQ ID NO: 720; or selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 164, SEQ ID NO: 166, SEQ ID NO: 168, SEQ ID NO: 170, SEQ ID NO: 172, SEQ ID NO: 174, SEQ ID NO: 176, and SEQ ID NO: 178; wherein the nucleic acid is operably linked to a heterologous promoter.

9. The recombinant vector of claim 8, wherein the nucleic acid has a polynucleotide sequence selected from the group consisting of SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, and SEQ ID NO: 719; or selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO: 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, and SEQ ID NO: 177.

10. A host cell comprising a recombinant vector of claim 8.

11. (canceled)

12. A method of producing a lignocellulose degradation enzyme, the method comprising culturing a cell that comprises a recombinant vector of claim 8 under conditions in which the enzyme is produced.

13. The method of claim 12, wherein the lignocellulose degradation enzyme is a glycohydrolase.

14. (canceled)

15. The method of claim 12, wherein the cell expresses at least one other recombinant lignocellulose degradation enzyme.

16. The method of claim 12, further comprising a step of recovering the lignocellulose degradation enzyme from the medium in which the cell is cultured or from a lysate of the cell.

17. A method for degrading a cellulosic biomass, the method comprising contacting the cellulosic biomass with a composition comprising a recombinant lignocellulose degradation enzyme of claim 1.

18. The method of claim 17, wherein the lignocellulose degradation enzyme is encoded by a polynucleotide having a nucleic acid sequence selected from the group consisting of SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, SEQ ID NO: 205, SEQ ID NO: 207, SEQ ID NO: 209, SEQ ID NO: 211, SEQ ID NO: 213, SEQ ID NO: 215, SEQ ID NO: 217, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 233, SEQ ID NO: 235, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 255, SEQ ID NO: 257, SEQ ID NO: 259, SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, SEQ ID NO: 287, SEQ ID NO: 289, SEQ ID NO: 291, SEQ ID NO: 293, SEQ ID NO: 295, SEQ ID NO: 297, SEQ ID NO: 299, SEQ ID NO: 301, SEQ ID NO: 303, SEQ ID NO: 305, SEQ ID NO: 307, SEQ ID NO: 309, SEQ ID NO: 311, SEQ ID NO: 313, SEQ ID NO: 315, SEQ ID NO: 317, SEQ ID NO: 319, SEQ ID NO: 321, SEQ ID NO: 323, SEQ ID NO: 325, SEQ ID NO: 327, SEQ ID NO: 329, SEQ ID NO: 331, SEQ ID NO: 333, SEQ ID NO: 335, SEQ ID NO: 337, SEQ ID NO: 339, SEQ ID NO: 341, SEQ ID NO: 343, SEQ ID NO: 345, SEQ ID NO: 347, SEQ ID NO: 349, SEQ ID NO: 351, SEQ ID NO: 353, SEQ ID NO: 355, SEQ ID NO: 357, SEQ ID NO: 359, SEQ ID NO: 361, SEQ ID NO: 363, SEQ ID NO: 365, SEQ ID NO: 367, SEQ ID NO: 369, SEQ ID NO: 371, SEQ ID NO: 373, SEQ ID NO: 375, SEQ ID NO: 377, SEQ ID NO: 379, SEQ ID NO: 381, SEQ ID NO: 383, SEQ ID NO: 385, SEQ ID NO: 387, SEQ ID NO: 389, SEQ ID NO: 391, SEQ ID NO: 393, SEQ ID NO: 395, SEQ ID NO: 397, SEQ ID NO: 399, SEQ ID NO: 401, SEQ ID NO: 403, SEQ ID NO: 405, SEQ ID NO: 407, SEQ ID NO: 409, SEQ ID NO: 411, SEQ ID NO: 413, SEQ ID NO: 415, SEQ ID NO: 417, SEQ ID NO: 419, SEQ ID NO: 421, SEQ ID NO: 423, SEQ ID NO: 425, SEQ ID NO: 427, SEQ ID NO: 429, SEQ ID NO: 431, SEQ ID NO: 433, SEQ ID NO: 435, SEQ ID NO: 437, SEQ ID NO: 439, SEQ ID NO: 441, SEQ ID NO: 443, SEQ ID NO: 445, SEQ ID NO: 447, SEQ ID NO: 449, SEQ ID NO: 451, SEQ ID NO: 453, SEQ ID NO: 455, SEQ ID NO: 457, SEQ ID NO: 459, SEQ ID NO: 461, SEQ ID NO: 463, SEQ ID NO: 465, SEQ ID NO: 467, SEQ ID NO: 469, SEQ ID NO: 471, SEQ ID NO: 473, SEQ ID NO: 475, SEQ ID NO: 477, SEQ ID NO: 479, SEQ ID NO: 481, SEQ ID NO: 483, SEQ ID NO: 485, SEQ ID NO: 487, SEQ ID NO: 489, SEQ ID NO: 491, SEQ ID NO: 493, SEQ ID NO: 495, SEQ ID NO: 497, SEQ ID NO: 499, SEQ ID NO: 501, SEQ ID NO: 503, SEQ ID NO: 505, SEQ ID NO: 507, SEQ ID NO: 509, SEQ ID NO: 511, SEQ ID NO: 513, SEQ ID NO: 515, SEQ ID NO: 517, SEQ ID NO: 519, SEQ ID NO: 521, SEQ ID NO: 523, SEQ ID NO: 525, SEQ ID NO: 527, SEQ ID NO: 529, SEQ ID NO: 531, SEQ ID NO: 533, SEQ ID NO: 535, SEQ ID NO: 537, SEQ ID NO: 539, SEQ ID NO: 541, SEQ ID NO: 543, SEQ ID NO: 545, SEQ ID NO: 547, SEQ ID NO: 549, SEQ ID NO: 551, SEQ ID NO: 553, SEQ ID NO: 555, SEQ ID NO: 557, SEQ ID NO: 559, SEQ ID NO: 561, SEQ ID NO: 563, SEQ ID NO: 565, SEQ ID NO: 567, SEQ ID NO: 569, SEQ ID NO: 571, SEQ ID NO: 573, SEQ ID NO: 575, SEQ ID NO: 577, SEQ ID NO: 579, SEQ ID NO: 581, SEQ ID NO: 583, SEQ ID NO: 585, SEQ ID NO: 587, SEQ ID NO: 589, SEQ ID NO: 591, SEQ ID NO: 593, SEQ ID NO: 595, SEQ ID NO: 597, SEQ ID NO: 599, SEQ ID NO: 601, SEQ ID NO: 603, SEQ ID NO: 605, SEQ ID NO: 607, SEQ ID NO: 609, SEQ ID NO: 611, SEQ ID NO: 613, SEQ ID NO: 615, SEQ ID NO: 617, SEQ ID NO: 619, SEQ ID NO: 621, SEQ ID NO: 623, SEQ ID NO: 625, SEQ ID NO: 627, SEQ ID NO: 629, SEQ ID NO: 631, SEQ ID NO: 633, SEQ ID NO: 635, SEQ ID NO: 637, SEQ ID NO: 639, SEQ ID NO: 641, SEQ ID NO: 643, SEQ ID NO: 645, SEQ ID NO: 647, SEQ ID NO: 649, SEQ ID NO: 651, SEQ ID NO: 653, SEQ ID NO: 655, SEQ ID NO: 657, SEQ ID NO: 659, SEQ ID NO: 661, SEQ ID NO: 663, SEQ ID NO: 665, SEQ ID NO: 667, SEQ ID NO: 669, SEQ ID NO: 671, SEQ ID NO: 673, SEQ ID NO: 675, SEQ ID NO: 677, SEQ ID NO: 679, SEQ ID NO: 681, SEQ ID NO: 683, SEQ ID NO: 685, SEQ ID NO: 687, SEQ ID NO: 689, SEQ ID NO: 691, SEQ ID NO: 693, SEQ ID NO: 695, SEQ ID NO: 697, SEQ ID NO: 699, SEQ ID NO: 701, SEQ ID NO: 703, SEQ ID NO: 705, SEQ ID NO: 707, SEQ ID NO: 709, SEQ ID NO: 711, SEQ ID NO: 713, SEQ ID NO: 715, SEQ ID NO: 717, and SEQ ID NO: 719.

19. The method of claim 17, wherein the composition is a cell culture medium into which the lignocellulose degradation enzyme has been secreted by cells expressing the enzyme.

20. (canceled)

21. The method of claim 17, wherein the lignocellulose degradation enzyme is a glycohydrolase.

22. (canceled)

23. A composition comprising a cellulase and a recombinant lignocellulose degradation enzyme of claim 1.

24. (canceled)

25. A composition of claim 23, wherein the lignocellulose degradation enzyme is a glyocoside hydrolase lignocellulose degradation enzyme and further, wherein the cellulase is different from the glycoside hydrolase lignocellulose degradation enzyme.

26. The composition of claim 25, wherein the glyocoside hydrolase lignocellulose degradation enzyme is set forth in Table 2.

27.-28. (canceled)
